(19) United States
(12) Patent Application Publication
(10) Pub. No.: US 2026/0290613 A1
(43) Pub. Date: Sep. 24, 2026

(54) METHODS AND SYSTEMS FOR ASSESSING POLYGENIC DISEASE RISK USING ENHANCER RNA REGULATORY VARIATIONS

(71) Applicant: Vanderbilt University, Nashville, TN (US)

(72) Inventors: Eric Gamazon, Nashville, TN (US); Ravi V. Shah, Nashville, TN (US)

(21) Appl. No.: 19/571,412

(22) Filed: Mar. 18, 2026

Related U.S. Application Data
(60) Provisional application No. 63/775,971, filed on Mar. 21, 2025.

(51) Int. Cl. G16H 50/30; G16B 20/20; G16B 40/20

(52) U.S. Cl. CPC G16H 50/30; G16B 20/20; G16B 40/20

(57) ABSTRACT

Disclosed are methods, systems, and non-transitory computer-readable media for assessing a subject's risk of developing a polygenic disease using genetic variation that regulates expression of enhancer RNAs (eRNAs). In various embodiments, genomic data for a subject are obtained and analyzed to determine whether the subject possesses one or more genetic variants associated with altered expression of at least one disease-associated eRNA, and the subject is classified as having an increased risk or a baseline risk. A disease risk score may be computed using a trained eRNA-based genetic risk model based at least in part on the presence or absence of the genetic variants. Genetically regulated expression (GReX) values for one or more eRNAs may be imputed from the genomic data using a trained tissue-specific predictive model and used to refine risk classification, optionally using disease-association parameters derived from transcriptome-wide association studies (TWAS) and/or Mendelian randomization analyses.

### RELATED APPLICATIONS

This application claims priority from U.S. Provisional Application Ser. No. 63/775,971 filed Mar. 21, 2025, the entire disclosure of which is incorporated herein by this reference.

### GOVERNMENT INTEREST

This invention was made with government support under HG010718, awarded by the National Institute of Health. The government has certain rights in the invention.

### TECHNICAL FIELD

The presently disclosed subject matter generally relates to computational genomics and bioinformatics, including methods and systems for analyzing genomic variation and regulatory features to assess disease risk and infer functional genomic relationships. In particular, certain embodiments of the presently disclosed subject matter relate to computing-based assessment of polygenic disease risk using genetic variants that regulate enhancer RNA (eRNA) expression, including tissue-specific imputation of genetically regulated expression (GReX) and use of association frameworks such as transcriptome-wide association studies (TWAS) and Mendelian randomization.

### INTRODUCTION

Enhancers are widely recognized as important regulators of gene expression, including through recruitment of DNA-binding proteins and establishment of chromatin conformation. While enhancer action has been studied extensively, the biological functions of eRNAs have been comparatively underexplored.

A persistent challenge in human genetics is that many trait-associated variants identified by genome-wide association studies (GWAS) lie in non-coding regions, complicating efforts to connect statistical associations to causal regulatory mechanisms and downstream biological effects. Approaches that prioritize canonical gene expression and promoter-centric regulation may fail to capture regulatory features operating distally or in a tissue-dependent manner, particularly where enhancer activity is highly context specific.

Another limitation in current practice is the incomplete correspondence between trait-associated loci and expression quantitative trait loci (eQTLs) derived from canonical gene expression. Prior work has described systematic differences between genetic effects discovered for complex traits and those mapped for gene expression, contributing to a so-called “colocalization gap” in which many GWAS signals do not align with known eQTLs under available mapping strategies and tissue sampling.

Relatedly, transcriptome-wide association studies (TWAS) have been used to implicate genes or regulatory features associated with disease risk, but TWAS findings can be sensitive to tissue selection, model training datasets, and the degree to which transcript features are genetically regulated. Prior eRNA-focused analyses have been described as limited in tissue scope (e.g., modeling enhancer expression in a small number of brain regions), which can constrain discovery of tissue-specific associations and limit interpretability across broader biological contexts.

In parallel, mapping three-dimensional genome organization and enhancer-gene relationships remains technically and computationally demanding. Generating high-resolution chromatin contact maps (e.g., Hi-C-based maps) can be methodologically challenging due to the scale of the data, and existing approaches for predicting chromatin contacts have relied on features such as genomic distance, Hi-C measurements themselves, or one-dimensional epigenomic datasets, which may not fully represent tissue-dependent regulatory behavior.

Finally, while rich multi-omic datasets can clarify regulatory mechanisms, it is often impractical to collect comprehensive functional genomics data at population scale due to cost, time, and sample limitations. This constraint motivates heavy reliance on in silico inference from germline variation, but existing inference approaches may not adequately incorporate enhancer-derived transcripts or may be limited by training datasets and assay biases (including enrichment for particular transcript subclasses).

There remains a need in the art for robust, tissue-aware computational frameworks and data resources that improve mechanistic interpretation of non-coding genetic variation, including enhancer-associated transcriptional regulation, and that enable reliable linkage between genetic associations, regulatory features, chromatin organization, and clinically relevant risk stratification for complex traits and polygenic diseases.

### SUMMARY

The presently disclosed subject matter meets some or all of the above-identified needs, as will become evident to those of ordinary skill in the art after a study of information provided in this document.

This Summary describes several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter relates generally to computational methods and systems for evaluating the phenotypic consequences of non-coding regulatory variation, including assessing disease risk using genetic features linked to eRNAs. In various embodiments, genomic data from a subject are analyzed to determine whether the subject possesses one or more genetic variants that regulate expression of one or more disease-associated eRNAs, and the subject is classified as having an increased risk or a baseline risk of a polygenic disease based at least in part on the presence or absence of such variants.

In certain embodiments, the disclosed methods and systems compute a disease risk score using a trained eRNA-based genetic risk model, thereby enabling quantitative risk stratification that incorporates regulatory variation affecting eRNA expression. In some embodiments, disease-association parameters derived from transcriptome-wide association studies (TWAS) and/or Mendelian randomization analyses are used to weight the contribution of genetic variants and/or eRNA features to the disease risk score.

In certain embodiments, the disclosed methods and systems further impute genetically regulated expression (GReX) of eRNAs using trained tissue-specific predictive models, and use the imputed GReX values to refine disease risk classification. By modeling tissue-dependent genetic regulation of enhancer transcription, the disclosed approaches provide tissue-relevant features that may not be captured by approaches restricted to canonical gene expression alone.

In certain embodiments, the presently disclosed subject matter also provides computer-implemented methods and systems for predicting three-dimensional chromatin contact between genomic elements using genetically regulated expression. In some embodiments, GReX values for transcripts associated with two genomic elements, wherein at least one transcript is an eRNA, are applied to a trained contact prediction model to output a predicted chromatin contact frequency for enhancer-gene and/or enhancer-enhancer pairs. In some embodiments, predicted contact frequencies across multiple pairs are used to generate a chromatin contact map for a genomic region and/or to rank candidate regulatory relationships based at least in part on predicted contact frequency.

In certain embodiments, the disclosed subject matter further provides methods and systems for identifying causal eRNAs associated with traits by integrating genetic association data with eRNA expression prediction models. In some embodiments, an eRNA-based TWAS identifies candidate eRNAs (including tissue-eRNA associations), and Mendelian randomization is applied to assess whether genetically regulated expression of a candidate eRNA has a causal effect on a trait. In some embodiments, eQTL mapping and colocalization analysis are performed to determine whether a GWAS locus colocalizes with regulatory variation affecting a candidate eRNA, and an eRNA-target report is generated identifying one or more causal eRNAs, implicated tissues, supporting genetic variants, and/or colocalization results.

The disclosed subject matter overcomes shortcomings of prior technologies that focus primarily on canonical gene expression regulation and therefore may miss, or under-resolve trait mechanisms driven by distal and tissue-dependent regulatory variation in non-coding elements. In particular, by incorporating eRNA-linked regulatory features, the disclosed approaches improve mechanistic interpretability of genetic associations and provide tissue-specific inference that better reflects context-dependent regulatory biology.

As described herein, predictive models of eRNA expression were trained using whole-genome sequencing data and eRNA expression profiled across 49 cell and tissue types (FIG. 1A), enabling quantification of genetic control. Using a deep learning-based framework, pairwise genetically regulated expression was shown to predict three-dimensional chromatin contact frequency for enhancer-gene and enhancer-enhancer pairs (FIG. 1B), supporting a modulatory role in spatial chromatin organization. An eRNA-based TWAS of schizophrenia risk was then performed (FIG. 1C), identifying eRNA associations independent of canonical genes, with causality assessed using Mendelian randomization (FIG. 1D). Finally, this eRNA-based TWAS methodology was applied across the UK Biobank to generate a comprehensive reference resource of tissue-specific enhancer associations across the human phenome (FIG. 1E).

In some embodiments, the disclosed functionality is implemented as executable instructions stored on a non-transitory computer-readable medium and executed by one or more processors, including implementations that receive genomic data, impute genetically regulated eRNA expression, compute disease risk scores, and output disease risk classifications and/or predicted chromatin contact frequencies.

### BRIEF DESCRIPTION OF THE DRAWINGS

The features of the invention are set forth with particularity in the appended claims. A better understanding of the features and advantages of the presently disclosed subject matter will be obtained by reference to the following detailed description that sets forth illustrative embodiments, in which the principles of the invention are used, and the accompanying drawings of which:

FIG. 1A-1E. Study workflow. FIG. 1A. In silico genetic models of genetically regulated expression (GReX) of eRNAs and canonical genes were trained using whole genome sequencing data and corresponding expression profiling generated by the GTEx Consortium. FIG. 1B. Using the trained models, GReX of eRNAs and canonical genes was imputed across 72,828 BioVU samples. Mean predicted expression values were used to train deep learning models of three-dimensional contact frequencies observed in Hi-C contact matrices. FIG. 1C. eRNA-based and canonical-gene-based TWAS of schizophrenia (SCZ) were run. FIG. 1D. Genome-wide significant TWAS associations were tested for causality using Mendelian randomization. This allowed for identification of loci with a causal SCZ-associated eRNA, gene, or both and to explore the potential underlying mechanisms by which they could influence disease risk. FIG. 1E. The eRNA-based TWAS models were applied the UK Biobank, generating a comprehensive resource of tissue-specific eRNA associations on a phenome-wide scale. FIG. 1F. eQTL mapping and colocalization analysis was performed across nearly 1 million independent GWAS associations in the UK Biobank to identify the proportion of signals explained by an eRNA eQTL versus a canonical gene eQTL.

FIG. 2A-2D. Training neural GReX-based neural network models using root mean squared error (RMSE) as the selection criterion. FIG. 2A. The optimal model for whole blood consisted of two hidden layers, each with a size of 130. The ReLU6 activation function was used in the hidden layers. Weights in the hidden layers were initialized using a Kaiming uniform distribution, while those in the output layer were initialized with zeros. FIG. 2B. The model was trained for 50 epochs using a batch size of 60. The NAdam optimizer was implemented with a learning rate of 0.002. The optimal model achieved an RMSE of 61.80 in the validation set, corresponding to an R² of 0.20. The model using R² as the selection criterion achieved a comparable validation R² of 0.22. FIG. 2C. The optimal model for cerebellum consisted of two hidden layers, each with a size of 90. The Softsign activation function was used in the hidden layers. Weights in the hidden layers were initialized using a normal distribution, while those in the output layer were initialized with zeros. FIG. 2D. The model was trained for 80 epochs using a batch size of 120. The Adagrad optimizer was implemented with a learning rate of 0.2. The optimal model achieved an RMSE of 25.47 in the validation set, corresponding to an R² of 0.39. The model using R² as the selection criterion achieved a comparable validation R² of 0.37.

FIG. 3. Distribution of transcript length among the 14,471 unique eRNAs included in the GReX models.

FIG. 4A-4D. Comparison of eRNA-based models with canonical-gene-based models in 49 GTEx tissues. FIG. 4A. Across all tissues, the eRNA-based models included a substantially lower proportion of imputable transcripts than the canonical gene-based models. FIG. 4B. Mean prediction R² varied by tissue type, with some tissue models having a higher predictive performance for eRNAs and others having a higher performance for canonical genes. FIG. 4C. The mean MAF of variants included in the models did not vary between eRNAs and canonical genes. FIG. 4D. The eRNA-based models, across all tissues, yielded a higher mean number of SNPs per gene transcript than the canonical-gene-based models.

FIG. 5A-5D. Distributions of whole blood training and test data for the deep learning-based models of chromatin contact frequency. FIG. 5A. Mean genetically regulated expression (GReX) values for the upstream transcript imputed in BioVU for whole blood. FIG. 5B. Mean genetically regulated expression (GReX) values for the downstream transcript imputed in BioVU for whole blood. FIG. 5C. Genomic distance (in base pairs) between the transcripts in each pair. FIG. 5D. Chromatin contact frequencies between whole blood transcript pairs (from Hi-C in K562).

FIG. 6A-6D. Distributions of cerebellum test data for the deep learning-based models of chromatin contact frequency. FIG. 6A. Mean genetically regulated expression (GReX) values for the upstream transcript imputed in BioVU for cerebellum. FIG. 6B. Mean genetically regulated expression (GReX) values for the downstream transcript imputed in BioVU for cerebellum. FIG. 6C. Genomic distance (in base pairs) between the transcripts in each pair. FIG. 6D. Chromatin contact frequencies between cerebellum transcript pairs (from Hi-C in astrocytes of the cerebellum).

FIG. 7A-7D. Training curves for non-linear models in whole blood. FIG. 7A. A series of polynomial regression models was fit, testing a range of degree values (1-10). The best-performing model (R²=0.02) used a degree of 7. FIG. 7B. A series of random forest regression models was fit, testing different numbers of trees (10-100). The best-performing model (R²=0.04) used 40 trees. FIG. 7C. A series of support vector regression models was fit, testing different epsilon values (0.1-5.0). The best-performing model (R²=0.00) used an epsilon value of 5. FIG. 7D. A series of gradient boosting regression models was fit, testing different numbers of boosting stages (10-300). The best-performing model (R²=0.08) used 290 boosting stages.

FIG. 8A-8D. Training curves for non-linear models in cerebellum. FIG. 8A. A series of polynomial regression models was fit, testing a range of degree values (1-10). The best-performing model (R²=0.08) used a degree of 8. FIG. 8B. A series of random forest regression models was fit, testing different numbers of trees (10-100). The best-performing model (R²=0.13) used 100 trees. FIG. 8C. A series of support vector regression models was fit, testing different epsilon values (0.1-5.0). The best-performing model (R²=0.01) used an epsilon value of 5. FIG. 8D. A series of gradient boosting regression models was fit, testing different numbers of boosting stages (10-300). The best-performing model (R²=0.13) used 290 boosting stages.

FIG. 9A-9F. Distributions of K562 nuclear run-on datasets for the baseline model of chromatin contact frequency. FIG. 9A. Expression values for the upstream transcript of each enhancer-enhancer or enhancer-gene pair prior to normalization. FIG. 9B. Expression values for the downstream transcript of each enhancer-enhancer or enhancer-gene pair prior to normalization. FIG. 9C. Expression values for the upstream transcript of each enhancer-enhancer or enhancer-gene pair after log 1p normalization. FIG. 9D. Expression values for the downstream transcript of each enhancer-enhancer or enhancer-gene pair after log 1p normalization. FIG. 9E. Genomic distance (in base pairs) between the transcripts in each pair. FIG. 9F. Chromatin contact frequencies between cerebellum transcript pairs (from Hi-C in K562).

FIG. 10A-10B. Nuclear run-on-based baseline model. FIG. 10A. A grid search across 13 hyperparameters was used to achieve the optimal model architecture. FIG. 10B. The neural network regressor was trained for 90 epochs, achieving a mean prediction R² of 0.23 across the validation folds and 0.27 in the independent test set.

FIG. 11A-11D. Whole blood-based deep learning model predicts three-dimensional contact frequency but lacks cross-tissue portability. FIG. 11A. A grid search across 13 hyperparameters was used to achieve the optimal model architecture. FIG. 11B. The neural network regressor was trained for 90 epochs, achieving a mean prediction R² of 0.22 in both the validation folds and the independent test set derived from whole blood. In a second test tissue, cerebellum, a prediction R² of 0.01 was achieved. FIG. 11C. Contact frequency predictions in the whole blood test set (denoted by shading) relative to the input GReX of the upstream and downstream transcripts (x- and y-axes, respectively). FIG. 11D. SHAP values computed for the transcript pairs in the test set. The relative mean contributions of the upstream and downstream transcripts were 39.33% and 60.67%, respectively.

FIG. 12A-12D. Deep learning model with tissue-specific eRNA and canonical gene GReX as features predicts three-dimensional contact frequency. FIG. 12A. Grid search across 13 hyperparameters was used to find the optimal model architecture (see Examples). FIG. 12B. The neural network was trained in cerebellum GReX and contact frequency data for 50 epochs, achieving a mean prediction R2 of 0.37 across the validation folds and 0.38 in the independent test set. Within a second tissue type not previously seen by the model, whole blood, a prediction R2 of 0.18 was observed, which shows some cross-tissue portability. FIG. 12C. Contact frequency prediction in the cerebellum test set (denoted by shading) as a function of the GReX of the upstream and downstream transcript levels (x and y axes, respectively). The two-dimensional GReX space for contact pairs is constrained to lie in the shaded region. FIG. 12D. SHAP values representing the relative mean contributions of the upstream and downstream transcripts to contact frequency predictions, which was found to be 46.94% and 53.06%, respectively.

FIG. 13A-13D. Correlation of deep learning model predictions with linear genomic distance. Using Pearson correlation, a weak negative correlation between genomic distance and predicted contact frequency was observed for both the model trained on GReX and Hi-C data from whole blood (FIG. 13A) and the cerebellum-trained model (FIG. 13B). A weak negative correlation between genomic distance and observed contact frequency was also observed for both the model trained on GReX and Hi-C data from whole blood (FIG. 13C) and the cerebellum-trained model (FIG. 13D).

FIG. 14A-14D. eRNA-based TWAS of schizophrenia with corresponding GWAS and canonical gene TWAS associations. TWAS was performed using the summary statistics from a logistic regression-based GWAS of schizophrenia (plotted on the bottom half of FIGS. 14A and 14C). The outer horizontal lines in these plots indicate the Bonferroni-corrected genome-wide significance threshold (p=5×10⁻⁸), and the middle horizontal lines indicates suggestive significance (p=1×10⁻⁶). Because the eRNA-based TWAS (plotted on the upper half of each plot) included 40,749 eRNA-tissue pairs (14,471 unique transcripts), the Bonferroni-corrected p-value threshold used for genome-wide significance was 1.23×10⁻⁶ and 3.46×10⁻⁶. Because the canonical-gene-based TWAS (plotted on the lower half of FIGS. 14B and 14D) included 344,814 gene-tissue pairs (26,138 unique transcripts), the Bonferroni-corrected p-value threshold used for genome-wide significance was 1.45×10⁻⁷ and 1.91×10⁻⁶ for suggestive significance. FIG. 14A. The full set of eRNA TWAS results for all 49 GTEx tissues plotted to mirror the GWAS results. FIG. 14B. eRNA-based and canonical-gene-based TWAS results across all 49 tissues. FIG. 14C. The eRNA-based TWAS results from 13 brain-derived tissues plotted against the GWAS results. FIG. 14D. Brain-based eRNA and canonical-gene TWAS results. FIG. 14E. Hi-C contact data (10 kb resolution) from primary astrocytes of the cerebellum was used to identify enhancer-gene contacts in the brain. Significant transcribed enhancer and canonical gene associations from TWAS in physical contact were tested for putative causality. The heatmap on the left depicts all Hi-C contacts (from chr20) prior to filtering, with the intensity scale corresponding to normalized contact frequency. The right-hand plot shows a subset of those contacts between a transcribed enhancer and canonical gene.

FIG. 15A-15B. Epigenomic patterns inform possible mechanisms by which causal eRNAs influence risk for SCZ. FIG. 15A. If causal eRNAs influence SCZ risk via direct mediation of enhancer-gene interactions, strong enrichment of either RAD21 and SMC3 or CTCF would be expected. However, only a small proportion of eRNAs were enriched for these features, and no evidence of contact with a causal gene was observed. FIG. 15B. If causal eRNA expression plays a role in maintaining an open chromatin state, enrichment of ATAC-seq and DNase-seq peaks, as well as EP300 and H3K27ac, in addition to depletion of H3K27me3, would be expected. Enrichment for at least one of these marks was observed in 74% of causal SCZ-associated eRNAs in the brain.

FIG. 16A-16B. Functional analysis of causal eRNAs suggests that these transcripts mediate SCZ risk via contact-independent rather than contact-dependent mechanisms. FIG. 16A. It was hypothesized that if the causal eRNAs influenced SCZ risk via contact-dependent mechanisms, positive enrichment of cohesin complex sub-components RAD21 and SMC3 or CTCF would be observed. If the causal eRNAs influenced SCZ risk via contact-independent mechanisms, it was hypothesized that one should observe enrichment of chromatin accessibility, histone marks H3K27ac and H3K4me1, and histone acetyltransferase EP300, as well as a depletion of H3K27me3. An absence of RAD21, SMC3, and CTCF would also be expected. The functional analyses supported a contact-independent model. Expected enrichment is indicated with in FIG. 16A with “+” in circles, while expected depletion is indicated with “−” in circle. FIG. 16B. Transcription factor binding motif enrichment analysis using log likelihood ratio identified motifs for 135 unique factors that were significantly enriched in the sequences of the causal SCZ-associated eRNAs. Several of these TFs (EGR243-45, SOX1046,47, TCF4/ITF248,49, and SP450,51) have previously been implicated in SCZ. The significance threshold (FDR<0.05) is indicated with the dashed line.

FIG. 17A-17B. eRNA eQTLs are associated with complex traits across the phenome and help to explain 63% more GWAS signals than canonical gene eQTLs alone. FIG. 17A. Depicted are the top 50 heritable UK Biobank traits ranked by number of significant (p<2.60× 10⁻¹⁰) eRNA-tissue TWAS associations. The shading intensity for each phenotype corresponds to the p-value of its most significant eRNA association. FIG. 17B. Using a Bayesian framework, 18,815 genome-wide significant (p<5×10⁻⁸) GWAS signals were identified within the UK Biobank that only colocalized (posterior probability >0.7) with an eRNA eQTL, representing a 63% increase over the number of independent associations that can be explained by canonical gene eQTLs alone.

FIG. 18. Jaccard similarity indices assessing overlap between eRNA eQTLs and canonical gene eQTLs. Across all tissues (n=49), a mean Jaccard similarity index of 0.05 was observed, indicating minimal overlap between eRNA and canonical gene eQTLs. The lowest Jaccard statistic was observed in brain putamen (0.03), while the highest was observed in testis (0.09).

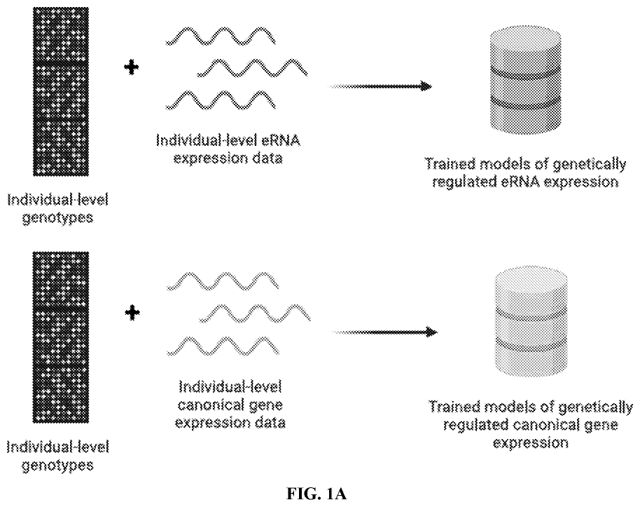

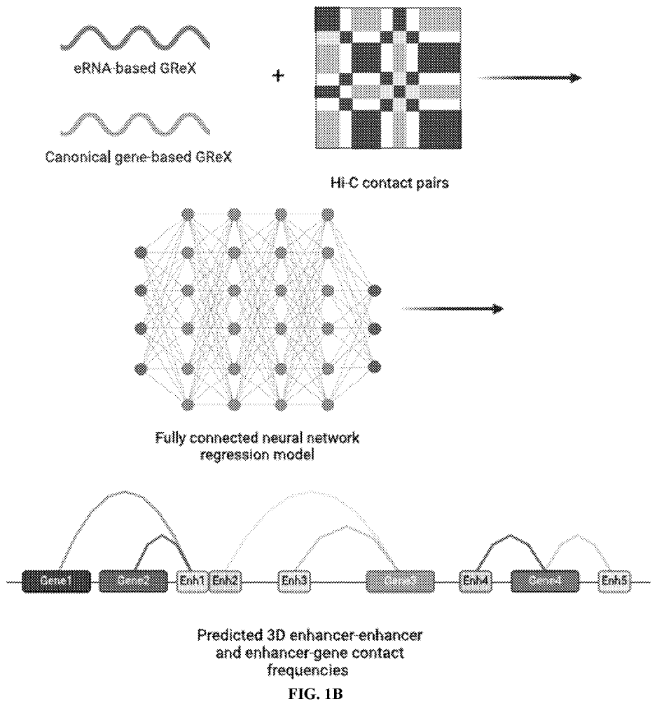

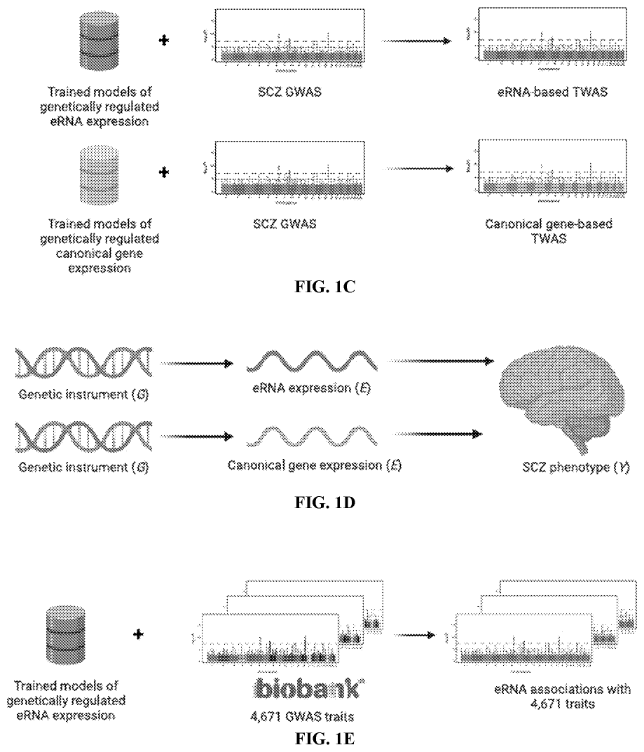

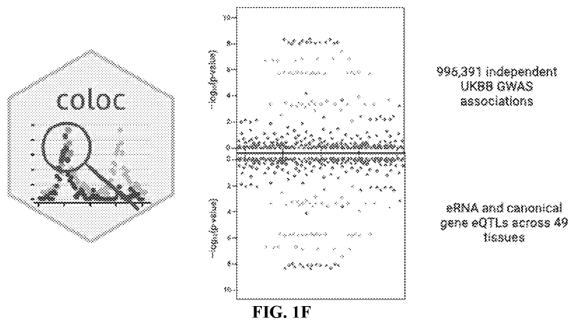

### DESCRIPTION OF EXEMPLARY EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently disclosed subject matter includes methods for assessing a subject's risk of developing a polygenic disease by analyzing genetic variation that regulates expression of one or more eRNAs associated with the polygenic disease. A genetic variant that regulates expression of an eRNA is variation in a genomic sequence that is associated with altered expression of one or more eRNAs. Such variants may include, for example, single nucleotide polymorphisms (SNPs), insertions, deletions, copy number variations, or structural variants, and may exert regulatory effects directly or indirectly, including through cis- or trans-acting mechanisms. In exemplary embodiments, genomic data obtained from a subject are evaluated to determine whether the subject possesses one or more alleles at genetic variants that influence expression of disease-associated eRNAs, and the subject is classified as having an increased risk or a baseline risk of the polygenic disease based at least in part on the presence or absence of such alleles.

In some embodiments, classifying the subject as having an increased risk or a baseline risk of the polygenic disease further comprises computing a disease risk score for the subject using a trained eRNA-based genetic risk model. The trained model may be stored in a non-transitory computer-readable medium and may be configured to integrate information regarding the presence or absence of genetic variants that regulate expression of one or more disease-associated eRNAs. The subject may then be classified based at least in part on the computed disease risk score relative to one or more reference values or thresholds.

In some embodiments, the method further comprises imputing genetically regulated expression (GReX) values for a plurality of eRNAs from the subject's genomic data using a trained, tissue-specific predictive model. The imputed GReX values may represent predicted expression levels attributable to genetic regulation and may be used to refine or adjust the subject's risk classification for the polygenic disease.

In some embodiments, imputing genetically regulated eRNA expression comprises applying predictive models trained using whole-genome sequencing data in combination with eRNA expression datasets derived from a plurality of human tissues. Such training data may enable the predictive models to capture tissue-specific and disease-relevant patterns of genetic regulation of eRNA expression.

In some embodiments, classifying the subject comprises determining whether one or more imputed GReX values for disease-associated eRNAs meet or exceed predetermined thresholds associated with altered risk for the polygenic disease. The predetermined thresholds may be derived from population-level analyses, reference cohorts, or disease-association studies and may be used to distinguish increased risk from baseline risk classifications.

In some embodiments, the method further comprises computing a disease risk score using data derived from the subject's genomic data, wherein computing the disease risk score comprises weighting the presence or absence of genetic variants using disease-association parameters obtained from transcriptome-wide association studies (TWAS) and/or Mendelian randomization analyses. Such weighting may reflect the relative contribution of specific genetic variants or eRNAs to disease susceptibility.

In some embodiments, the polygenic disease comprises a disorder selected from neuropsychiatric disorders, cardiometabolic disorders, hematologic traits or disorders, and sensory function disorders. The disclosed methods may be applied across multiple disease categories in which risk is influenced by the combined effects of multiple genetic variants regulating eRNA expression or the effect of the eRNA expression itself.

In some embodiments, the polygenic disease is selected from schizophrenia, hypertension, manifestations of mania or irritability, hearing impairment, platelet distribution width abnormality, neutrophil percentage abnormality, hemoglobin concentration abnormality, lymphocyte count abnormality, leukocyte count abnormality, cholesterol-related disorders, and obesity-related disorders. These examples are illustrative and do not limit the scope of the disclosed subject matter.

In some embodiments, the polygenic disease comprises schizophrenia, and the genetic variants that regulate expression of one or more eRNAs comprise genetic variants that regulate expression of ENSR00000013481 and/or ENSR00000032851. In such embodiments, the subject is classified as having an increased risk of schizophrenia when at least one disease-predisposing allele at a genetic variant regulating expression of ENSR00000013481 or ENSR00000032851 is present in the subject's genomic data, and as having a baseline risk of schizophrenia when none of such genetic variants are present. ENSR00000013481 and ENSR00000032851 are specific eRNA loci identified by their respective reference identifiers, including genomic regions and corresponding transcripts associated with regulatory activity and genetically regulated expression relevant to schizophrenia risk. References to these eRNAs include sequence variants, allelic forms, transcript isoforms, and functionally equivalent eRNA transcripts arising from the same or overlapping enhancer regions.

In some embodiments, classifying the subject as having an increased or baseline risk of schizophrenia further comprises computing a disease risk score for schizophrenia using a trained eRNA-based genetic risk model. The trained model may be stored in a non-transitory computer-readable medium and configured to evaluate the presence or absence of genetic variants that regulate expression of ENSR00000013481 and/or ENSR00000032851. The subject's schizophrenia risk classification may be determined based at least in part on the computed disease risk score.

In some embodiments, the method further comprises imputing GReX values for ENSR00000013481 and/or ENSR00000032851 from the subject's genomic data using a trained brain-specific predictive model. The imputed GReX values may be used to refine or adjust the subject's schizophrenia risk classification, including by providing quantitative measures of genetically regulated eRNA expression relevant to schizophrenia.

In some embodiments, classifying the subject comprises determining whether one or more imputed GReX values for ENSR00000013481 and/or ENSR00000032851 meet or exceed predetermined thresholds associated with altered schizophrenia risk. Such thresholds may be derived from population-level schizophrenia studies or reference cohorts and may be used to distinguish increased risk from baseline risk.

In some embodiments, computing the predicted genetically regulated expression level of ENSR00000013481 and/or ENSR00000032851 is performed using an eRNA expression prediction model trained on brain tissue transcriptome data.

In some embodiments, the brain tissue transcriptome data comprise expression data from one or more brain regions selected from cortex, hippocampus, amygdala, substantia nigra, cerebellum, and cerebellar hemisphere. Expression data from different brain regions may be used individually or in combination to capture region-specific regulatory effects relevant to schizophrenia.

In some embodiments, refining the schizophrenia risk classification further comprises correlating predicted genetically regulated expression of ENSR00000013481 with expression of VPS45 and correlating predicted genetically regulated expression of ENSR00000032851 with expression of NT5C2. Such correlations may reflect downstream regulatory relationships relevant to schizophrenia susceptibility. VPS45 refers to the vacuolar protein sorting 45 gene and its corresponding transcripts and protein products, including allelic variants and isoforms, and encompasses measurements of expression level thereof. VPS45 is a downstream gene whose expression may be associated with genetically regulated expression of ENSR00000013481. NT5C2 refers to the cytosolic 5′-nucleotidase II gene and its corresponding transcripts and protein products, including allelic variants and isoforms, and encompasses measurements of expression level thereof. NT5C2 is referenced herein as a downstream gene whose expression may be associated with genetically regulated expression of ENSR00000032851.

In some embodiments, the method further comprises assessing expression of VPS45 and/or NT5C2 in a biological sample obtained from the subject. The subject's schizophrenia risk classification may be increased when reduced VPS45 expression is observed in a subject possessing a genetic variant regulating ENSR00000013481 and/or when reduced NT5C2 expression is observed in a subject possessing a genetic variant regulating ENSR00000032851.

In some embodiments, the method further comprises computing a disease risk score for schizophrenia using data derived from the subject's genomic data, wherein computing the disease risk score comprises weighting the presence or absence of genetic variants regulating ENSR00000013481 and/or ENSR00000032851 using disease-association parameters obtained from transcriptome-wide association studies (TWAS) and/or Mendelian randomization analyses. Such weighting may reflect inferred causal or statistically significant contributions to schizophrenia risk.

In some embodiments, the presently disclosed subject matter includes a non-transitory computer-readable medium storing instructions that, when executed by one or more processors, cause the one or more processors to perform the methods described herein, including receiving genomic data, imputing genetically regulated eRNA expression, computing disease risk scores, and classifying polygenic disease risk.

In some embodiments, the presently disclosed subject matter includes a non-transitory computer-readable medium storing instructions that, when executed by one or more processors, cause the one or more processors to perform any of the methods described herein. The instructions may implement one or more steps of obtaining genomic data, analyzing genetic variants regulating eRNA expression, computing disease risk scores, imputing genetically regulated eRNA expression, and classifying a subject's disease risk.

In some embodiments, the presently disclosed subject matter includes a computer-implemented system for assessing a subject's risk of developing a polygenic disease. The system comprises at least one processor and a non-transitory computer-readable storage medium storing instructions that, when executed by the at least one processor, cause the system to receive genomic data for the subject, analyze the genomic data to determine whether the subject possesses one or more genetic variants that regulate expression of one or more eRNAs, and classify the subject as having an increased risk or a baseline risk of the polygenic disease based at least in part on whether such genetic variants are present. Such classification of the subject refers to a computer-implemented determination assigning the subject to one of at least two risk categories based on analysis performed by the system. The classification may be performed automatically and may be updated or refined as additional data are evaluated.

In some embodiments, the instructions stored in the non-transitory computer-readable storage medium further cause the system to compute a disease risk score for the polygenic disease using a trained eRNA-based genetic risk model. The disease risk score may be computed based at least in part on the presence or absence of genetic variants that regulate expression of one or more disease-associated eRNAs, and the system may classify the subject as having an increased risk or a baseline risk of the polygenic disease based on the computed disease risk score.

In some embodiments, the instructions further cause the system to impute GReX values for a plurality of eRNAs from the subject's genomic data using a trained tissue-specific predictive model. The imputed GReX values may represent predicted expression levels attributable to genetic regulation and may be used by the system to inform or refine the subject's disease risk classification.

In some embodiments, imputing the genetically regulated eRNA expression comprises applying predictive models trained using whole-genome sequencing data in combination with eRNA expression datasets derived from a plurality of human tissues. Such training data may enable the system to capture tissue-dependent patterns of genetic regulation relevant to polygenic disease risk.

In some embodiments, classifying the subject as having an increased risk or a baseline risk of the polygenic disease comprises determining whether one or more imputed GReX values for disease-associated eRNAs meet or exceed predetermined thresholds associated with altered disease risk. The thresholds may be derived from population-level analyses, reference cohorts, or disease-association studies and may be applied by the system to distinguish increased risk from baseline risk classifications.

In some embodiments, the instructions further cause the system to compute a disease risk score for the polygenic disease using data derived from the subject's genomic data, wherein computing the disease risk score comprises weighting the presence or absence of genetic variants using disease-association parameters obtained from transcriptome-wide association studies (TWAS) and/or Mendelian randomization analyses. Such weighting may reflect statistical association or inferred causal contributions of genetic features to disease susceptibility.

In some embodiments, the polygenic disease assessed by the system is selected from neuropsychiatric disorders, cardiometabolic disorders, hematologic traits or disorders, and sensory function disorders. The system may be configured to assess disease risk across multiple categories of polygenic diseases using shared or disease-specific predictive models.

In some embodiments, the polygenic disease assessed by the system is selected from schizophrenia, hypertension, manifestations of mania or irritability, hearing impairment, platelet distribution width abnormality, neutrophil percentage abnormality, hemoglobin concentration abnormality, lymphocyte count abnormality, leukocyte count abnormality, cholesterol-related disorders, and obesity-related disorders. These examples are illustrative and do not limit the scope of the disclosed system.

In some embodiments, the system is configured to assess a subject's risk of schizophrenia. In such embodiments, the instructions stored in the non-transitory computer-readable storage medium further cause the system to determine, from the subject's genomic data, whether the subject possesses one or more genetic variants that regulate expression of eRNAs ENSR00000013481 and/or ENSR00000032851. The system may classify the subject as having an increased risk of schizophrenia when at least one such genetic variant is present and as having a baseline risk of schizophrenia when none of such genetic variants are present.

In some embodiments, the instructions further cause the system to compute a disease risk score for schizophrenia using a trained eRNA-based genetic risk model stored in the non-transitory computer-readable storage medium. The disease risk score may be computed based at least in part on the presence or absence of genetic variants that regulate expression of ENSR00000013481 and/or ENSR00000032851. The system may classify the subject as having an increased or baseline risk of schizophrenia based on the computed disease risk score.

In some embodiments, the instructions further cause the system to impute genetically regulated expression (GReX) values for eRNAs ENSR00000013481 and/or ENSR00000032851 from the subject's genomic data using a trained brain-specific predictive model. The system may refine the subject's schizophrenia risk classification based at least in part on the imputed GReX values.

In some embodiments, refining the subject's schizophrenia risk classification comprises determining whether the imputed GReX values for ENSR00000013481 and/or ENSR00000032851 meet or exceed predetermined thresholds associated with altered risk for schizophrenia. The system may use such thresholds to distinguish increased risk from baseline risk classifications.

In some embodiments, imputing the genetically regulated expression values for ENSR00000013481 and/or ENSR00000032851 is performed using an eRNA expression prediction model trained on brain tissue transcriptome data. Training the predictive model on brain-derived data may improve relevance of the imputed expression values to schizophrenia risk.

In some embodiments, the brain tissue transcriptome data comprise expression data from one or more brain regions selected from cortex, hippocampus, amygdala, substantia nigra, cerebellum, and cerebellar hemisphere. Data from multiple brain regions may be used individually or in combination to support imputation of genetically regulated eRNA expression.

In some embodiments, refining the subject's schizophrenia risk classification further comprises correlating the imputed genetically regulated expression of ENSR00000013481 with expression of VPS45 and correlating the imputed genetically regulated expression of ENSR00000032851 with expression of NT5C2. Such correlations may reflect downstream regulatory effects relevant to schizophrenia susceptibility.

In some embodiments, the instructions further cause the system to receive data indicative of expression levels of VPS45 and/or NT5C2 in a biological sample obtained from the subject. Such data reflects or correlates with expression levels of a gene or transcript, including quantitative or qualitative measures obtained directly or indirectly from biological samples. Such data may represent, correspond to, or be derived from measurements of gene or transcript expression. Such data may include direct expression measurements, normalized values, relative expression values, or processed outputs generated from expression assays. The system may increase the subject's schizophrenia risk classification when reduced VPS45 expression is indicated in a subject possessing a genetic variant that regulates expression of ENSR00000013481 and/or when reduced NT5C2 expression is indicated in a subject possessing a genetic variant that regulates expression of ENSR00000032851.

In some embodiments, the instructions further cause the system to compute a disease risk score for schizophrenia using data derived from the subject's genomic data, wherein computing the disease risk score comprises weighting the presence or absence of genetic variants that regulate expression of ENSR00000013481 and/or ENSR00000032851 using disease-association parameters obtained from transcriptome-wide association studies (TWAS) and/or Mendelian randomization analyses.

The presently disclosed subject matter further includes methods, including computer-implemented methods, for predicting chromatin contact between genomic elements using genetically regulated expression. In some embodiments, genomic data are obtained for a subject and used to impute GReX values for a first transcript associated with a first genomic element and a second transcript associated with a second genomic element, wherein at least one of the transcripts is an eRNA. The imputed GReX values are applied as inputs to a trained contact prediction model, which outputs a predicted chromatin contact frequency between the first and second genomic elements.

In some embodiments, the first and second genomic elements comprise an enhancer-gene pair. In other embodiments, the first and second genomic elements comprise an enhancer-enhancer pair.

In some embodiments, the trained contact prediction model comprises a neural network regression model. In some embodiments, the trained contact prediction model is tissue-specific, including models trained for whole blood or brain tissue. In some embodiments, a brain-specific model is trained using cerebellum-associated chromatin contact data.

In some embodiments, predicted chromatin contact frequencies for a plurality of genomic element pairs are used to generate a chromatin contact map for a genomic region. In some embodiments, candidate enhancer-gene regulatory relationships are ranked based at least in part on predicted chromatin contact frequency.

In some embodiments, imputing GReX values comprises applying predictive expression models trained using whole-genome sequencing data and transcript expression data from a plurality of tissues. In some embodiments, the applied GReX values correspond to an upstream transcript and a downstream transcript. In some embodiments, an indication is output that chromatin contact is tissue-dependent based on applying two or more tissue-specific trained contact prediction models.

In some embodiments, the presently disclosed subject matter includes a non-transitory computer-readable medium storing instructions that cause one or more processors to perform the chromatin contact prediction methods described herein. In some embodiments, a system is provided that is configured to perform such methods, including generating chromatin contact maps across multiple genomic element pairs.

The presently disclosed subject matter further includes methods, including computer-implemented methods, for identifying a causal eRNA associated with a trait. In some embodiments, genetic association data for a trait are obtained, along with one or more eRNA expression prediction models that map genetic variants to genetically regulated expression of eRNAs. An eRNA-based TWAS is performed to identify one or more candidate eRNAs associated with the trait.

In some embodiments, Mendelian randomization analysis is performed for at least one candidate eRNA to determine whether genetically regulated expression of the candidate eRNA has a causal effect on the trait. An indication that the candidate eRNA is causal may be output when a causality criterion is satisfied. In some embodiments, the genetic association data comprise genome-wide association study (GWAS) summary statistics.

In some embodiments, the eRNA expression prediction models are tissue-specific, and the eRNA-based TWAS identifies tissue-eRNA associations. In some embodiments, a canonical gene-based TWAS is additionally performed, and an eRNA association that is independent of canonical gene associations is identified.

In some embodiments, eQTL mapping is performed to identify one or more eRNA eQTLs for a candidate eRNA. In some embodiments, colocalization analysis is performed between a GWAS locus and an eRNA eQTL, and an indication is output that the GWAS locus colocalizes with regulatory variation affecting the candidate eRNA.

In some embodiments, an eRNA-target report is generated that includes one or more of an identifier of a causal eRNA, an implicated tissue, supporting genetic variants, or an indication of colocalization with an eRNA eQTL. In some embodiments, the trait comprises a polygenic disease.

In some embodiments, a system and/or non-transitory computer-readable medium is provided that is configured to perform the causal eRNA identification methods described herein.

The presently disclosed subject matter further includes methods for identifying a target gene regulated by a transcribed eRNA. In some embodiments, a transcribed enhancer associated with a trait is selected, the transcribed enhancer corresponding to an eRNA, and the enhancer is perturbed in a cell using a targeted perturbation agent. Expression of one or more genes is measured after perturbation, and a gene is identified as a target gene when its expression changes in response to the perturbation.

In some embodiments, the targeted perturbation agent comprises a CRISPR interference (CRISPRi) system. In some embodiments, gene expression is measured using RNA sequencing or quantitative PCR.

In some embodiments, chromatin contact data are evaluated to determine whether the transcribed enhancer and the target gene exhibit chromatin contact, and an indication is output that regulation is contact-independent when no chromatin contact is detected. In some embodiments, shared eQTL analysis is performed, and an indication is output that regulation is not explained by a shared eQTL when no shared eQTL is detected.

In some embodiments, an association record linking the eRNA to the target gene is stored in a database, wherein the association record is usable to refine a genetic risk model for the trait. In some embodiments, the trait comprises schizophrenia. In some embodiments, the identified target gene is used to select or prioritize a therapeutic intervention or candidate therapeutic target.

The methods and systems described herein may make use of biological and genomic datasets, including datasets comprising genomic variation, eRNA expression, chromatin accessibility, chromatin conformation, and transcriptional activity. Such datasets may be obtained from genomic and functional genomics databases maintained by public consortia, research organizations, or other sources. These datasets provide suitable inputs for training, evaluating, and validating the models described herein. In addition, equivalent datasets of the same general type, whether public or proprietary, may be used.

Publicly available data sets were used, in certain implementations, to train, evaluate, and validate the models described herein. Enhancer RNA genetically regulated expression models, transcriptome-wide association study results, Mendelian randomization results, and colocalization results generated in connection with the present work were deposited in public repositories, including Zenodo (accession code 14557414). Trained chromatin contact frequency prediction models were deposited in public repositories, including GitHub and Zenodo. In addition, previously published genetically regulated expression models for canonical genes were obtained from public repositories and used in connection with the analyses described herein.

Nuclear run-on transcription data from the K562 cell line were obtained from the ENCODE Data Portal (accession code ENCSR363AKK). Hi-C chromatin contact datasets for K562 cells and cerebellar astrocytes were obtained from the 4D Nucleome Data Portal (accession codes 4DNFI18UHVRO and 4DNFIWCAQUIK). In addition, ChIP-seq and chromatin accessibility datasets for neuronal cell lines were obtained from the ENCODE Data Portal, including datasets corresponding to histone modifications, chromatin-associated proteins, and chromatin accessibility (accession codes ENCFF362OBM, ENCFF277NRX, ENCFF580GTZ, ENCFF051ZRW, ENCFF756DQA, ENCFF244QKO, ENCFF654KAP, ENCFF716JUM, and ENCFF752OZB).

Curated transcription start site coordinate annotations were obtained from the refTSS database, and quantified eRNA expression data were obtained from the HeRA resource. Genotype data from human cohorts were accessed subject to applicable data use agreements, including controlled-access repositories.

Equivalent datasets of the same general type may be substituted, and the methods and systems described herein are not limited to use with any particular dataset, repository, accession identifier, or dataset version.

Methods, devices, and materials similar or equivalent to those described herein may be used in the practice or testing of the presently disclosed subject matter, and representative methods, devices, and materials are described herein.

While the terms used herein are believed to be well understood by those of ordinary skill in the art, certain definitions are set forth to facilitate explanation of the presently disclosed subject matter.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUBMB Joint Commission on Biochemical Nomenclature (See, iubmb.qmul.ac.uk/).

Unless defined otherwise, all technical and scientific terms used herein have the same meaning as is commonly understood by one of skill in the art to which the invention(s) belong.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

A “biological sample” refers to any sample obtained from a subject that contains biological material suitable for assessing gene or transcript expression, including blood, tissue, cells, or derivatives thereof.

The term “classifying,” when used herein in connection with a subject, refers to assigning the subject to one or more risk categories, including baseline risk or increased risk, based at least in part on analysis of genomic data and optionally additional derived metrics.

“Baseline risk” refers to a reference level of risk for a polygenic disease corresponding to a population average, control cohort, or other predefined comparator group, and does not require absence of all genetic risk factors.

“Increased risk” refers to a level of risk for a polygenic disease that is greater than a baseline risk, as determined using one or more genetic, computational, or statistical criteria, including presence of disease-associated genetic variants, imputed expression values, or computed risk scores.

“Risk classification” refers to categorizing a subject according to relative risk of developing a polygenic disease, including baseline risk and increased risk, based on one or more genetic, computational, or molecular criteria.

“Increase the subject's risk classification” refers to adjusting a subject's assigned disease risk category to a higher risk level relative to a prior classification. Increasing risk classification does not require a specific numerical change and may include movement from baseline risk to increased risk or from a lower to a higher risk tier.

“Refining” a risk classification refers to modifying, adjusting, or further specifying an initial disease risk assessment using additional data, analyses, or derived metrics, including imputed expression values or downstream gene expression measurements.

“Reduced” or “increased” expression refers to an expression level of a gene or transcript that is lower or higher, respectively, than a reference level, including a population average, control cohort, baseline value, or predetermined threshold.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

“Correlating” refers to assessing a relationship or association between two or more measured or predicted values, including statistical, computational, or qualitative relationships. Correlation does not require a particular statistical method or threshold and may include direct or indirect associations.

“Computer-implemented classification” refers to assigning a subject to one or more categories using operations performed by a computer system based on analysis of input data and optionally derived metrics, without requiring human judgment at the time of classification.

A “computer-implemented system” refers to a system comprising one or more computing devices configured to perform one or more operations using computer-executable instructions. A computer-implemented system may include hardware, software, firmware, or any combination thereof, and may operate locally, remotely, or in a distributed computing environment.

A “computer-readable storage medium” or “storage medium” refers to a physical medium capable of storing instructions or data in a non-transitory form, including memory devices, storage devices, or other tangible media.

The term “receiving,” when used in connection with a computer-implemented system receiving data, refers to acquiring data by a system or processor from any source, including direct input, data transmission, data retrieval from memory, or access to data generated or stored by another system or entity.

A “genome-wide association study (GWAS)” refers to an analysis that evaluates associations between genetic variants across the genome and a phenotype or disease outcome.

A “transcriptome-wide association study (TWAS)” refers to an analysis that evaluates associations between genetically regulated expression of transcripts and a phenotype or disease outcome.

“Mendelian randomization” refers to an analytical framework that uses genetic variants as instrumental variables to infer causal relationships between a molecular feature and a disease or trait.

A “non-transitory computer-readable medium” refers to a tangible storage medium capable of storing instructions executable by one or more processors, including memory, storage devices, or other physical media, and excludes transitory signals.

A “processor” refers to any programmable device capable of executing instructions, including a central processing unit (CPU), graphics processing unit (GPU), microprocessor, controller, or other logic circuitry. A processor may operate alone or in combination with one or more additional processors.

“Instructions” refer to machine-readable commands, code, or program logic that, when executed by one or more processors, cause the processor(s) to perform specified operations. Instructions may be implemented in software, firmware, or a combination thereof.

As used herein, “disease-associated eRNA” refers to an eRNA whose genetically regulated expression level is statistically associated with risk, susceptibility, or manifestation of a disease or trait, including a polygenic disease, as determined by one or more population-level analyses, such as GWAS, TWAS, or Mendelian randomization analyses.

“Disease-association parameters” refer to numerical or categorical values reflecting statistical associations between genetic features and disease risk, including effect sizes, weights, coefficients, or probabilities derived from population-level analyses.

A “disease risk score” refers to a quantitative or semi-quantitative value representing a subject's predicted risk of developing a disease, computed using genomic data and optionally imputed expression values, model parameters, or disease-association parameters.

“Weighting” refers to assigning relative values or contributions to individual genetic variants or features when computing a disease risk score. Weighting may be based on statistical association, inferred causality, or other disease-relevant parameters.

An “enhancer RNA” (eRNA) refers to an RNA transcript that is transcribed from a genomic enhancer region and is associated with regulatory activity affecting gene expression. eRNAs may be transcribed in a tissue-specific or context-dependent manner and may include transcripts that are non-coding, non-polyadenylated, polyadenylated, unidirectional, bidirectional, spliced, unspliced, long, or short. eRNAs may function through contact-dependent or contact-independent regulatory mechanisms and may influence chromatin state, transcription factor occupancy, or gene expression.

“Genetically regulated expression” (GReX) refers to an expression level of a transcript that is attributable to genetic variation, independent of non-genetic influences. GReX values may be predicted, imputed, or generated using predictive models trained on reference datasets comprising genotype and expression data.

“Genomic data” refers to data derived from a biological sample of a subject that provide information regarding the subject's genetic sequence or variation. Genomic data may include, without limitation, whole-genome sequencing data, whole-exome sequencing data, targeted sequencing data, genotyping array data, or any combination thereof, and may be provided in raw, processed, or annotated form. The term “obtaining,” when used in connection with genomic data, refers to acquiring genomic data directly or indirectly, including by generating the data from a biological sample, receiving the data from a third party, retrieving the data from a database, or accessing previously generated genomic data. The term “analyzing,” when used in connection with genomic data, refers to performing one or more computational, statistical, or algorithmic operations to identify, infer, or determine the presence or absence of one or more genetic variants, regulatory features, or derived metrics from the genomic data.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

A “polygenic disease” refers to a disease, disorder, condition, or trait for which risk or susceptibility is influenced by the combined effects of multiple genetic variants, including variants that regulate expression of one or more eRNAs, and optionally environmental or contextual factors. Polygenic diseases may include, for example, neuropsychiatric, cardiometabolic, hematologic, and sensory function disorders. A neuropsychiatric disorder refers to a disorder affecting neurological and/or psychiatric function, including disorders characterized by cognitive, emotional, or behavioral disturbances. Schizophrenia is an example of a neuropsychiatric disorder characterized by disturbances in thought, perception, emotion, and behavior. Additional examples of neuropsychiatric disorders are manifestations of mania or irritability. A cardiometabolic disorder refers to a disorder affecting cardiovascular and/or metabolic function, including conditions influenced by genetic regulation of metabolic or vascular pathways. Examples of cardiometabolic disorders include hypertension, cholesterol-related disorders, and obesity-related disorders. A hematologic trait or disorder refers to a measurable blood-related characteristic or disease, including traits or conditions involving blood cells, blood components, or hematopoietic processes. Examples of hematologic traits or disorders include platelet distribution width abnormality, neutrophil percentage abnormality, hemoglobin concentration abnormality, lymphocyte count abnormality, and leukocyte count abnormality. A sensory function disorder refers to a disorder affecting one or more sensory modalities, including hearing, vision, or other sensory systems. An example of a sensory function disorder is hearing impairment.

A “predictive model” or “trained model” refers to a computational model that has been generated or parameterized using training data and is configured to infer, estimate, or predict an output value from one or more input variables. Examples of predictive models include statistical models, machine-learning models, and combinations thereof and may be tissue-specific or disease-specific.

A “trained eRNA-based genetic risk model” refers to a predictive model that has been trained using training data to assess disease risk based at least in part on genetic features related to eRNAs. The trained eRNA-based genetic risk model is configured to receive, as input, genomic data, imputed GReX values of one or more eRNAs, or combinations thereof, and to output a disease risk score indicative of a subject's risk of developing a disease. The trained eRNA-based genetic risk model may comprise a statistical model, a machine-learning model, or a combination thereof, and may incorporate disease-association parameters derived from TWAS, Mendelian randomization analyses, or related population-level analyses. Disease-association parameters obtained from TWAS and/or Mendelian randomization analyses refer to quantitative or qualitative measures reflecting statistical association or inferred causality between genetically regulated expression of transcripts and polygenic disease risk, including effect sizes, weights, coefficients, or probability estimates.

A “tissue-specific predictive model” refers to a predictive model trained using transcriptome data derived from one or more specified human tissues and configured to impute or predict genetically regulated expression of one or more transcripts in a tissue-dependent manner. Tissue-specific predictive models may account for regulatory, genetic, or expression features characteristic of the tissue(s) on which the model is trained and may be used to infer expression levels relevant to disease risk in those tissues. An example of a tissue-specific predictive model is a “brain-specific predictive model,” which refers to a predictive model trained using transcriptome data derived from one or more brain tissues or brain regions and configured to impute genetically regulated expression of one or more transcripts in a manner relevant to neurological or neuropsychiatric phenotypes.

“Tissue transcriptome data” refers to gene or transcript expression data obtained from a specified tissue or tissue type, including bulk tissue data or cell-type-specific data, and may include RNA sequencing or other expression profiling data. Tissue transcriptome data may be derived from one or more human tissues and may be used to train predictive models of genetically regulated expression in a tissue-dependent manner. An example of tissue transcriptome data is “brain tissue transcriptome data” refers to gene or transcript expression data obtained from brain tissue samples, including bulk tissue or cell-type-specific data, and may include RNA sequencing or other expression profiling data derived from one or more brain regions. A “brain region” refers to a distinct anatomical or functional region of the brain. Exemplary brain regions include, without limitation, cortex, hippocampus, amygdala, substantia nigra, cerebellum, and cerebellar hemisphere.

The presently disclosed subject matter is further illustrated by the following specific but non-limiting examples. The following examples may include compilations of data that are representative of data gathered at various times during the course of development and experimentation related to the present invention.

### EXAMPLES

### Example 1: GTEx Project Data

Genetic data from deceased human individuals from the GTEx Project were included in this research. Protected GTEx Project data (for example, genotype and RNA-seq data) are available via an access request to dbGaP accession no. phs000424.v8.p2.

### Example 2: In Silico Genetic Models of eRNA Expression

The Human enhancer RNA Atlas (HeRA) (hanlab.uth.edu/HeRA/) is a publicly available resource of eRNA expression values processed from raw GTEx RNA-seq reads. These expression values were first normalized using the first five principal components, the first five PEER covariates, age, sex, and sequencing platform. Next, models were trained for each of the 49 GTEx cell and tissue types using an implementation of PrediXcan provided by MR-JTI, using whole genome sequencing (WGS) and the corresponding quantified eRNA expression data generated from 507 donors. PrediXcan models of canonical gene expression had been previously trained using the implementation provided in the MR-JTI repository and were retrieved from Zenodo under accession code 3842289 (JTI).

### Example 3: Training Set for Nuclear Run-On-Based Chromatin Contact Prediction

Because both eRNAs and canonical genes are 5′-capped, a K562 GRO-cap dataset enriched for capped RNAs was obtained from the ENCODE Data Portal (accession number ENCSR363AKK) in bigWig format. Files quantifying transcription from the plus and minus strands were combined and converted to bedGraph format. Using bedtools (v2.30.0), transcripts in the nuclear run-on assay that overlapped with known human eRNAs annotated by the ENSEMBL, FANTOM5, or Roadmap consortia, or with a known GENCODE (v32) gene, were identified. Transcript counts annotated with the same eRNA or gene were combined. Prior to model training, the final expression values were log 1p-normalized.

### Example 4: Training Set for GReX-Based Chromatin Contact Prediction

Trained models of eRNA and canonical gene expression were utilized to impute GReX in 72,828 BioVU samples across all 49 cell and tissue types. The Bio VU data consisted of individuals of European ancestry (31,861 males, 40,584 females, and 383 with unknown sex) genotyped on the Illumina MEGA array, followed by genotype imputation using the HRC panel. Age among individuals in the sample ranged from 0-90 years, with a median age of 56 years.

High-resolution Hi-C datasets generated in the K562 leukemic cell line (accession number 4DNFI18UHVRO) and in astrocytes of the cerebellum (accession number 4DNFIWCAQUIK) were retrieved from the 4D Nucleome Data Portal (data.4dnucleome.org) in mcool format. Raw sequencing reads underwent initial processing, contact matrix aggregation, and normalization using the gold-standard Hi-C processing pipeline described at data.4dnucleome.org/resources/data-analysis/hi_c-processing-pipeline. The Hi-C dataset representing whole blood (K562) included 907,136,828 filtered reads, while the cerebellum-based dataset included 428,475,763. Both datasets showed similar quality control metrics, such as cis/trans ratio, percentage of long-range intrachromosomal reads, and very good convergence. Contacts were normalized using the ICE (iterative correction and eigenvalue decomposition) algorithm.

Using the processed mcool file, Cooler (v0.8.2) was used to export contacts at 10 kb resolution with the cooler dump function, and an R script was written to convert these outputs to BEDPE format (available on GitHub). An initial set of contact regions was defined as those with one or more normalized contacts in each Hi-C dataset. These contact pairs were then filtered to a subset in which the respective 10 kb contact regions overlapped with either two annotated eRNAs or an eRNA and a canonical gene. The eRNA annotations were drawn from the same collection used by the HeRA and consisted of human eRNAs annotated by the ENSEMBL, FANTOM5, and Roadmap consortia. Canonical gene annotations were obtained from GENCODE (v32).

These contact data, together with mean eRNA and canonical gene GReX values for whole blood and cerebellum in BioVU, respectively, comprised the training set. The resulting dataset contained 85,630 unique genome-wide enhancer gene contact pairs in whole blood and 95,701 in cerebellum. Because the genomic distance distribution was left-skewed, these values underwent log 1p transformation.

### Example 5: Linear Regression Models of Chromatin Contact Frequency

Using scikit-learn (v1.2.2), two linear regression models were fit to predict contact frequency for enhancer-enhancer and enhancer-gene pairs: one based on genomic distance between the two elements and the other based on GReX of the respective eRNA and gene. An 80/20 train-test split was used, such that the whole blood training set consisted of 68,504 unique contact pairs and the held-out test set consisted of 17,126. The numbers of contact pairs in the cerebellum training and test sets were 76,560 and 19,141, respectively.

### Example 6: Non-Linear Models of Chromatin Contact Frequency

Scikit-learn (v1.2.2) was used to fit four non-linear models—polynomial regression, random forest regression, support vector regression, and gradient boosting regression—to predict enhancer-enhancer and enhancer-gene contact frequency using GReX data from both whole blood and cerebellum. An 80/20 train-validation split was applied, and grid search was used to select the optimal set of hyperparameters for each model.

For polynomial regression, grid search was conducted over degrees ranging from 1 to 10. For random forest regression, grid search was conducted over numbers of trees ranging from 10 to 100. For support vector regression, grid search was conducted over epsilon values ranging from 0.1 to 1. For gradient boosting regression, grid search was conducted over numbers of boosting stages ranging from 10 to 300. R² was used as the selection metric to identify the best-performing model for each approach.

### Example 7: Training Deep Learning Models Based on eRNA and Canonical Gene GreX

All deep learning models were trained on an Nvidia Tesla K80 GPU running CUDA. Using PyTorch (v1.13.1), two fully connected neural networks were trained to predict enhancer-gene contact frequency. The first model was trained using only mean genetically regulated eRNA and canonical gene expression imputed in BioVU, whereas the second model additionally incorporated the genomic distance between the two transcripts.

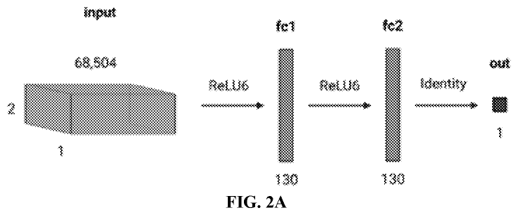

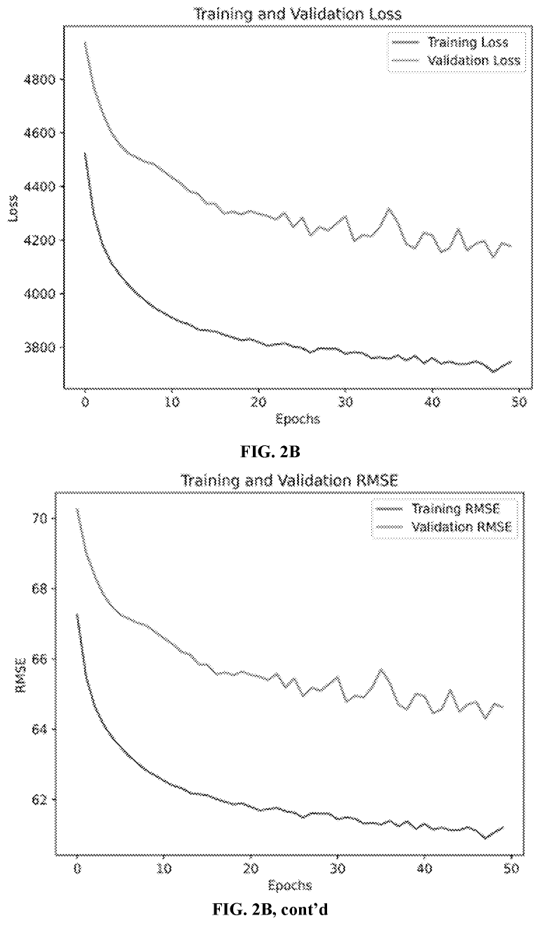

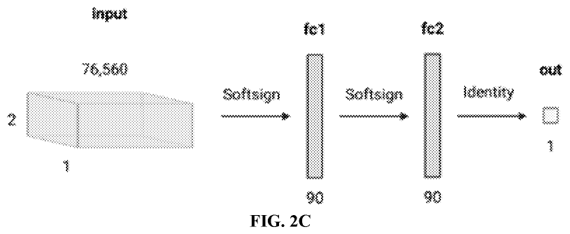

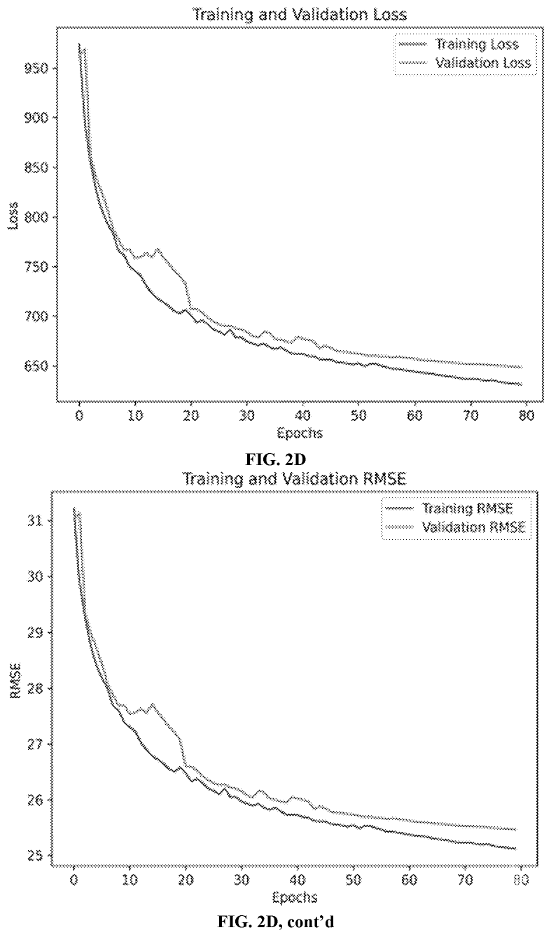

Both models were trained using five-fold cross-validation, and hyperparameters were tuned using grid search. An 80/20 train-test split was applied. During grid search, each network was iteratively optimized with respect to the number of hidden neurons, number of hidden layers, batch size, number of training epochs, optimizer and learning rate, weight initialization in the hidden and output layers, hidden-layer activation function, dropout, weight constraint, and L1 and L2 regularization parameters. Model selection was evaluated using both R² and root mean squared error (RMSE), and comparable results were observed (FIG. 2A-2D). Final model selection was based on maximization of predictive R².

Because this constituted a regression problem, mean squared error (MSE) was used as the loss function:

$$MSE = \frac{1}{n} {\sum }_{i = 1}^{n} {( {Y}_{i} - {\hat{Y}}_{i} )}^{2} \qquad ( 1 )$$

Here, n is equal to the number of data points, Yi represents the observed outcome, and Y′i represents the predicted outcome. A combination of L1 and L2 regularization was assumed. Predictive performance was evaluated using the R² metric:

$${R}^{2} = 1 - \frac{{\sum }_{i = 1}^{n} {( {\hat{Y}}_{i} - {Y}_{i} )}^{2}}{{\sum }_{i = 1}^{n} {( {Y}_{i} - \overline{Y} )}^{2}} \qquad ( 2 )$$

Here, n is equal to the number of data points, Yi represents the observed outcome, Y′i represents the predicted outcome, and y·i represents the mean of the observed outcomes.

### Example 8: SCZ PrediXcan TWAS

Using the eRNA models and previously published canonical gene expression models, S-PrediXcan, implemented in the MetaXcan GitHub repository (github.com/hakyimlab/MetaXcan), was utilized to perform summary statistics-based TWAS on a recent GWAS meta-analysis of schizophrenia. Multiple testing was accounted for using a Bonferroni correction. Thus, for the eRNA-based TWAS, the p-value threshold for genome-wide significance was

$$1.23 \times 1 {0}^{- 6} ( \frac{0.05}{40 , 749 eRNA - tissue pairs} ) ,$$

while the threshold for suggestive significance was

$$3.46 \times {10}^{- 6} ( \frac{0.05}{14 , 471 unique eRNAs} ) .$$

Meanwhile, for the canonical gene-based TWAS, genome-wide significant and suggestive p-value thresholds of

$$1.45 \times {10}^{- 7} ( \frac{0.05}{344 , 814 gene - tissue pairs} ) \\ and \\ 1.91 \times {10}^{- 6} ( \frac{0.05}{26 , 138 unique genes} ) ,$$

respectively, were used.

### Example 9: Mendelian Randomization of Significant SCZ TWAS Loci

Genome-wide significant associations from the respective eRNA-based and canonical gene-based TWAS were tested for causality using Mendelian randomization as implemented in MR-JTI (github.com/gamazonlab/MR-JTI). After each eRNA-tissue and canonical gene-tissue association was tested for causality, the causal associations identified were localized to their respective genetic loci to assess whether each locus contained a causal eRNA only, a causal canonical gene only, or both for SCZ.

### Example 10: Investigating Potential Functional Mechanisms of Causal eRNAs

ChIP-seq datasets generated in the SK—N—SH neuronal cell line were downloaded from the ENCODE Project data portal (www.encodeproject.org) in narrowPeak format for H3K27ac (ENCFF362OBM), H3K27me3 (ENCFF277NRX), H3K4me1 (ENCFF580GTZ), RAD21 (ENCFF051ZRW), SMC3 (ENCFF756DQA), CTCF (ENCFF244QKO), and EP300 (ENCFF654KAP). ATAC-seq (ENCFF716JUM) and DNase-seq (ENCFF752OZB) datasets were also retrieved for the same cell line. All datasets based on hg38 were lifted over to hg19 using liftOver.

### Example 11: Transcription Factor Binding Motif Enrichment

FIMO (v5.4.1; meme-suite.org/meme/doc/fimo.html) was used to test for enrichment of HOCOMOCO v11 human transcription factor binding motifs within the set of 34 causal SCZ-associated eRNAs in brain. Default parameters were used, and motifs with q-value (FDR)<0.05 were considered significantly enriched.

### Example 12: Phenome-Wide eRNA-Based TWAS in the UK Biobank

Using S-PrediXcan, the eRNA-based models were applied to GWAS summary statistics across 4,671 traits in the UK Biobank to generate a comprehensive resource of tissue-specific enhancer associations. A genome-wide significant threshold of p<2.60×10⁻¹⁰ was used, with the multiple testing threshold for genome-wide significance determined using Bonferroni correction:

$${p}_{eRNA} = \frac{0.05 / 41 , 086 eRNA - tissue pairs}{4671 complex traits} \qquad ( 3 )$$

### Example 13: Enhancer Perturbation Analysis for Disease-Associated eRNAs

A previously published enhancer perturbation dataset for the K562 cell line was obtained. Using CRISPRi, 5,920 human enhancers were perturbed, and resulting changes in gene expression were measured. Perturbed enhancer coordinates were converted to UCSC BED format, and enhancers overlapping known eRNAs annotated by the ENSEMBL, FANTOM5, or Roadmap consortia were identified using bedtools (v2.30.0; bedtools.readthedocs.io/En/latest/).

### Example 14: ERNA and Canonical Gene eQTL Mapping

The same genotype and normalized expression datasets used for eRNA- and canonical gene-based GReX model training were used to perform eQTL mapping. Cis-eQTLs were mapped using Matrix eQTL (v2.3).

### Example 15: Comparing eRNA eQTLs and Canonical Gene eQTLs

For each tissue, overlap between eRNA eQTLs and canonical gene eQTLs was assessed using the Jaccard similarity index. To assess distance from canonical genes, human transcription start site (TSS) coordinates from refTSS version 4.1 (reftss.riken.jp/datafiles/4.1/human/refTSS_v4.1_human_coordinate.hg38.bed.txt.gz) were used. Coordinates were lifted from hg38 to hg19 using liftOver (http://hgdownload.soe.ucsc.edu/admin/exe/liftOver.gz). Distances from the closest TSS for all eRNA and canonical gene eQTLs were computed using bedtools (v2.30.0; bedtools.readthedocs.io/en/latest/).

### Example 16: Colocalization of GWAS Signals with eRNA and Canonical Gene eQTLs

Across 4,671 complex traits in the UK Biobank, 996,391 independent, genome-wide significant (p<5×10⁻⁸) associations were identified. For each significant association, all GWAS SNPs and SNP eQTLs at the corresponding locus were used to perform colocalization analysis with coloc (v5.2.3). Colocalization was performed in each of the 49 GTEx tissues for both eRNA eQTLs and canonical gene eQTLs. A GWAS signal was considered to colocalize with an eQTL if the posterior probability was ≥0.7 in at least one tissue.

### Example 17: Genetic Models of eRNA Expression

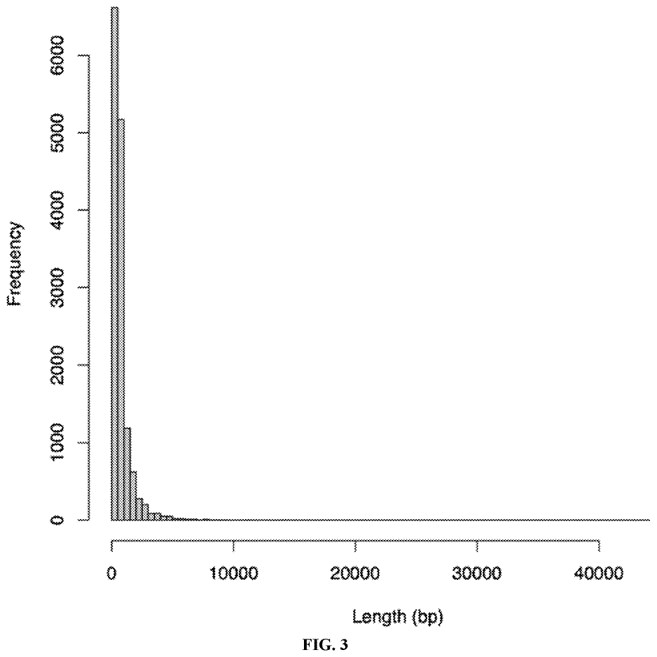

Models of genetically regulated eRNA expression were trained, encompassing a total of 14,471 transcribed enhancers profiled across 49 human cell and tissue types. Because eRNAs were quantified from RNA-seq data that underwent selection for polyadenylated transcripts prior to sequencing, 2D eRNAs, which are not polyadenylated, may have been underrepresented in the training dataset. Previous characterizations of eRNAs have described 2D transcripts as having a length below 2 kb, whereas 1D eRNAs are longer than 2 kb. Using transcript length, the proportion of eRNAs in the training dataset fitting the profile of a 2D eRNA versus a 1D eRNA was characterized. Of the 14,471 unique eRNA transcripts included in the trained models, 13,580 (93.84%) were less than 2 kb in length, while 891 (6.16%) had a length greater than or equal to 2 kb. The median eRNA transcript length was 550 bp (FIG. 3). These results suggest that, despite selection for polyadenylated transcripts, the vast majority of transcripts in the training set still exhibited characteristics typical of 2D eRNAs.

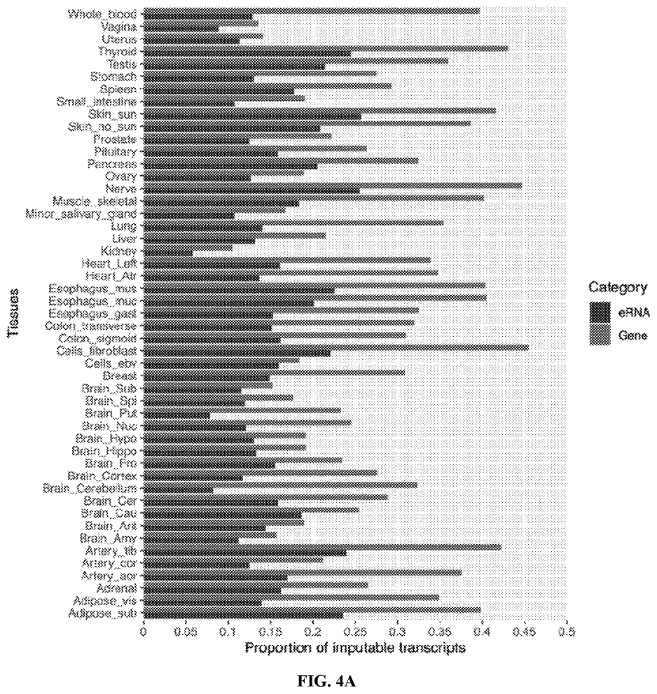

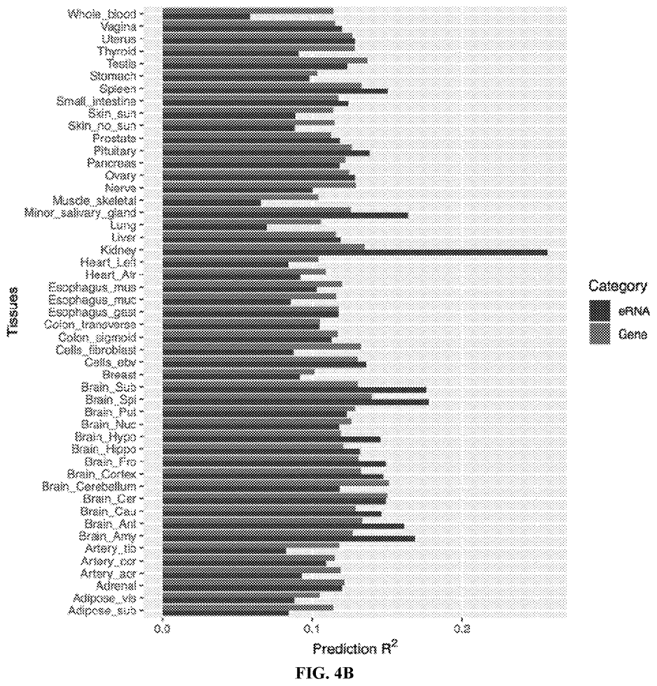

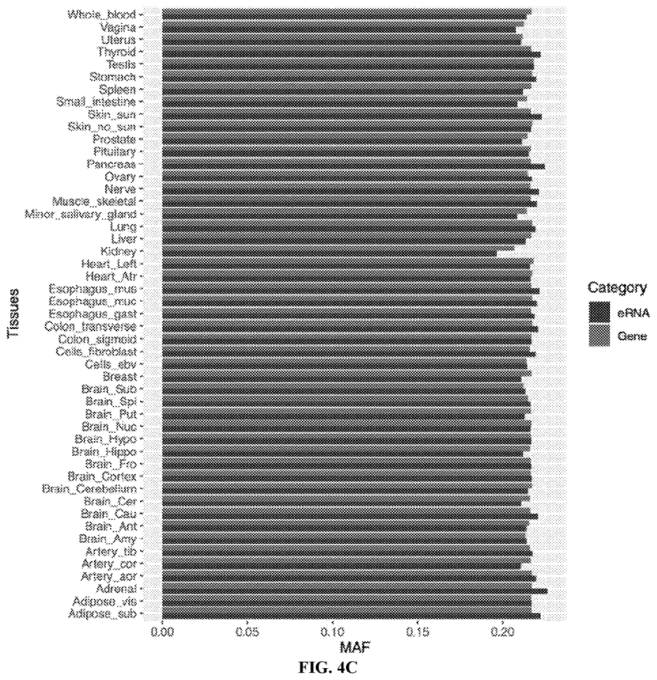

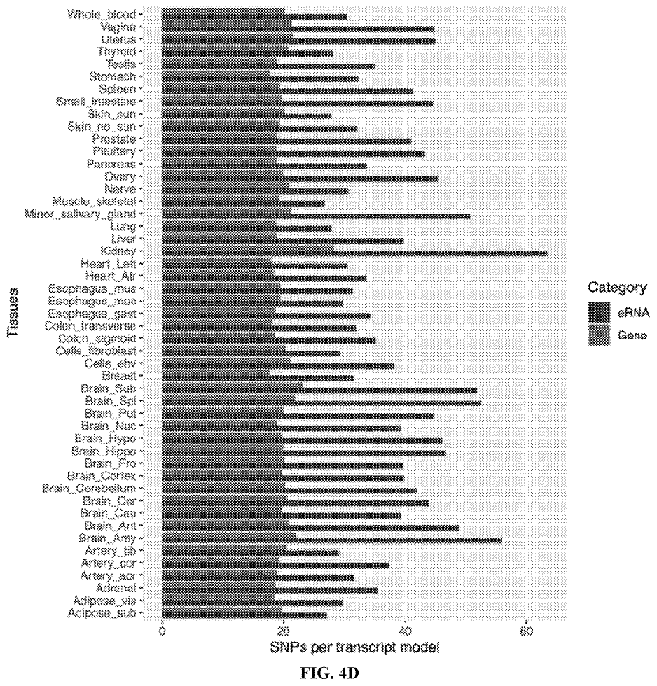

The resulting eRNA-based models were compared with an analogous set trained on canonical gene expression. The proportion of imputable transcripts, mean prediction R², mean minor allele frequency (MAF) of SNP features, and mean SNPs-per-transcript ratio were evaluated. For each tissue, a higher proportion of imputable canonical genes than imputable eRNAs was observed (FIG. 4A). Prediction R² showed greater cross-tissue variability (FIG. 4B). Across the 49 tissues, 21 tissues exhibited a higher mean prediction R² for eRNAs than for canonical genes. Although the mean MAF of model SNP features was essentially the same across all eRNA- and canonical-gene-based models (FIG. 4C), a significantly higher SNP-per-transcript ratio was observed for eRNA-based models across all cell and tissue types (mean 38.16 SNPs/eRNA versus 19.94 SNPs/canonical gene across all tissue models, p<2.2×10⁻¹⁶) (FIG. 4D).

### Example 18: Regression Modeling of GReX as a Predictor of Contact Frequency

Because enhancer-promoter interactions represent a critical mechanism of gene regulation, prediction of the functional consequences of genetic variation on chromatin contact has the potential to illuminate disease mechanisms. The extent to which genetically regulated eRNA and canonical gene expression predicted three-dimensional chromatin contact frequency was assessed.

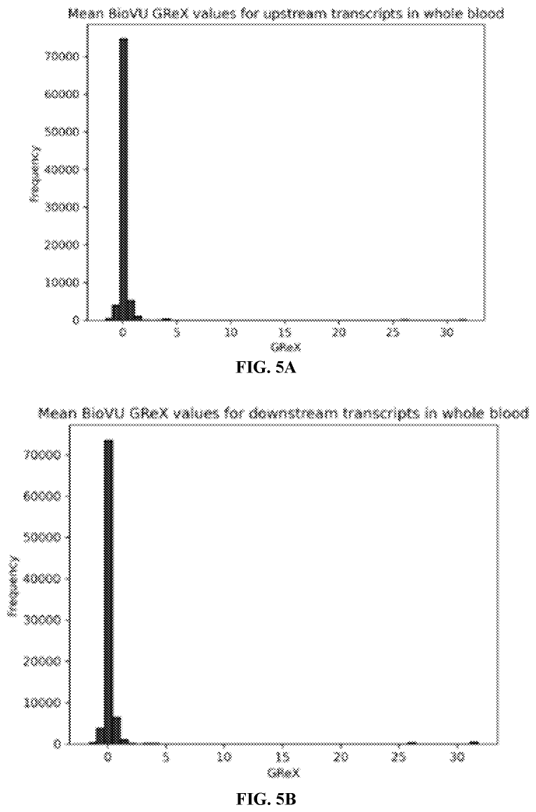

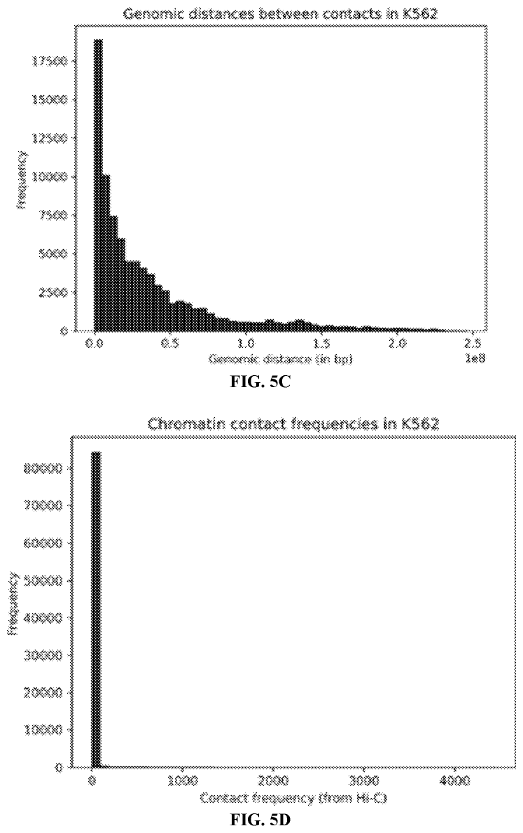

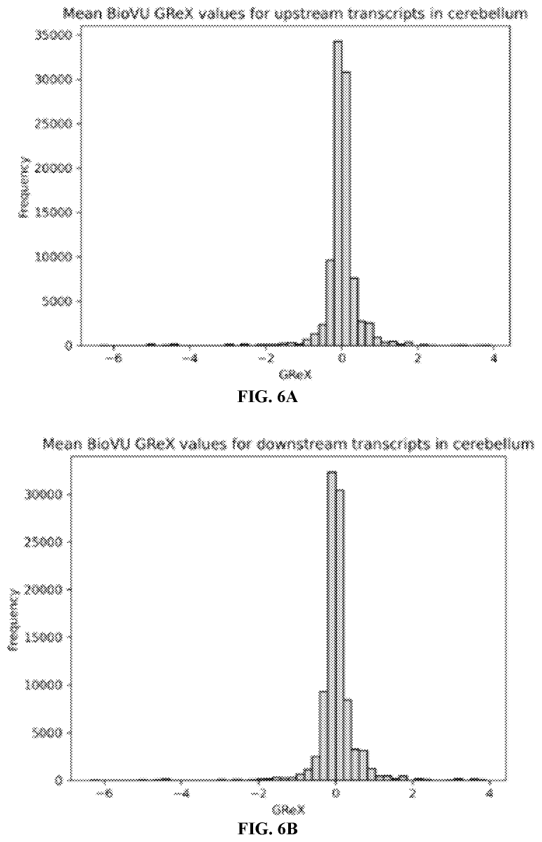

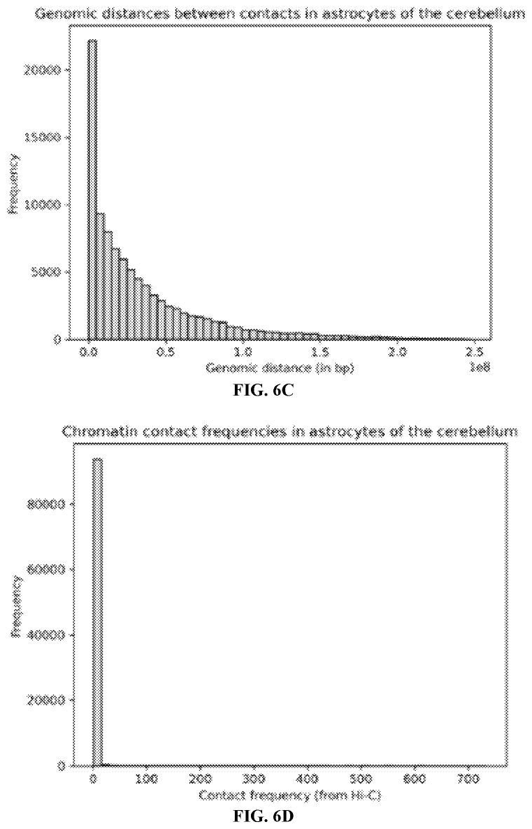

The eRNA and canonical gene models were applied to BioVU (N=72,828), Vanderbilt University Medical Center's DNA biobank linked to electronic health records, to impute sample-level GReX. Two high-resolution Hi-C datasets (4D Nucleome Data Portal; Methods) generated from the K562 leukemic cell line and primary astrocytes of the cerebellum were leveraged. Within the K562 dataset, 85,630 contact pairs overlapping enhancer-gene pairs with imputable expression (for each member transcript of the pair) were identified. Within the cerebellum dataset, 95,701 contact pairs for enhancer-gene pairs with corresponding imputable expression were identified. A training set was compiled for each tissue, consisting of mean GReX values (estimated in BioVU) for each transcript pair along with the normalized Hi-C contact frequency for the corresponding contact pair (FIGS. 5A-5D and 6A-6D).

As a baseline, a linear regression model was fit to predict 3D contact frequency for enhancer-enhancer or enhancer-gene pairs using mean predicted GReX of the corresponding transcripts in BioVU. An 80/20 train-test split was used to train the models. In whole blood, the training data consisted of 32, 133 enhancer-gene pairs (with the eRNA upstream and the gene downstream), 34,682 gene-enhancer pairs (with the gene upstream and the enhancer downstream), and 1,689 enhancer-enhancer pairs. In the cerebellum training data, these numbers were 37,710, 36,117, and 2,733, respectively. Poor performance was observed for the resulting linear model in each tissue (R²≈0).

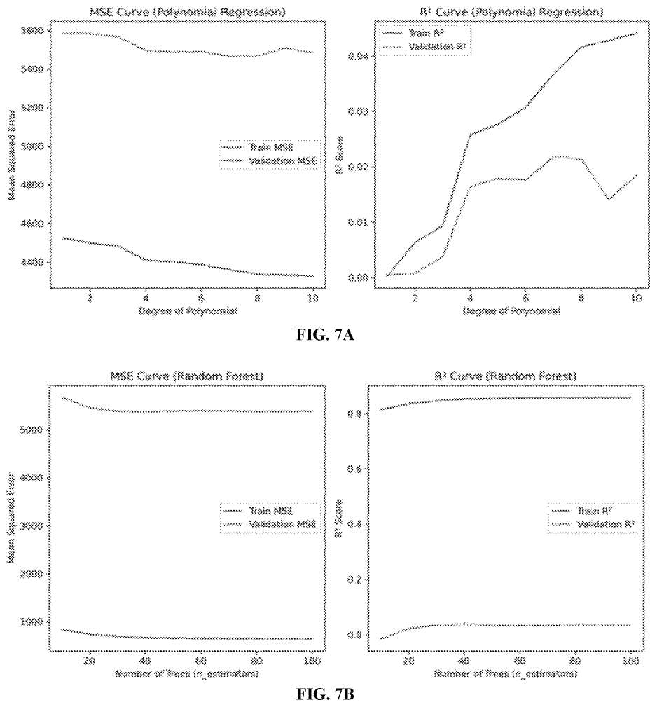

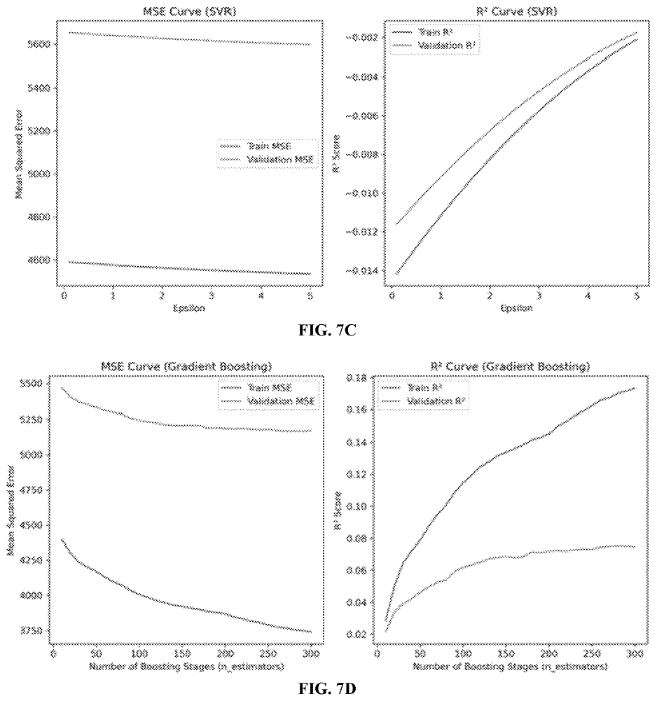

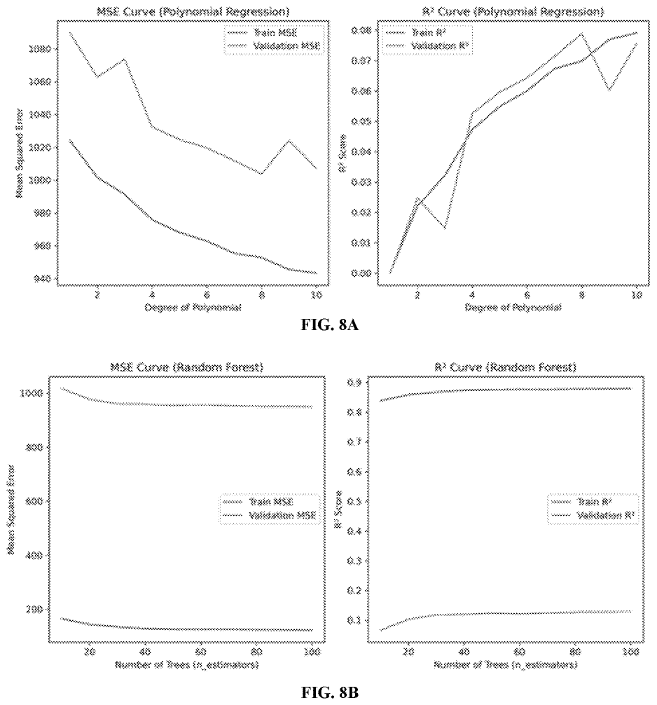

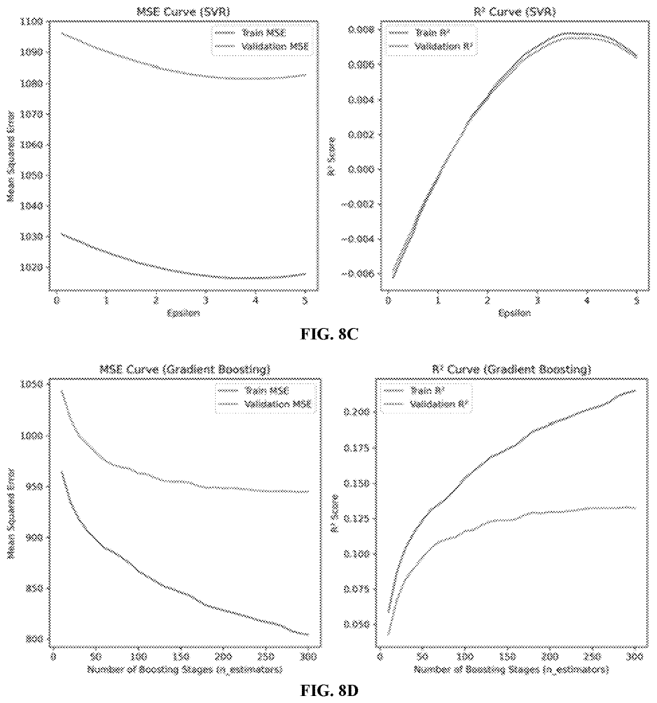

Given the low performance of the initial linear model, four non-linear models (polynomial regression, random forest regression, support vector regression, and gradient boosting regression) were trained to predict contact frequency using GReX data (FIGS. 7A-7D and 8A-8D). Among these non-linear models, gradient boosting regression yielded the highest performance in both whole blood (R²=0.08 using 290 boosting stages) and cerebellum (R²=0.13 using 290 boosting stages). The still-low performance supported the use of a more complex neural network-based approach.

### Example 19: Training a Baseline Contact Model Using Directly Assayed Expression

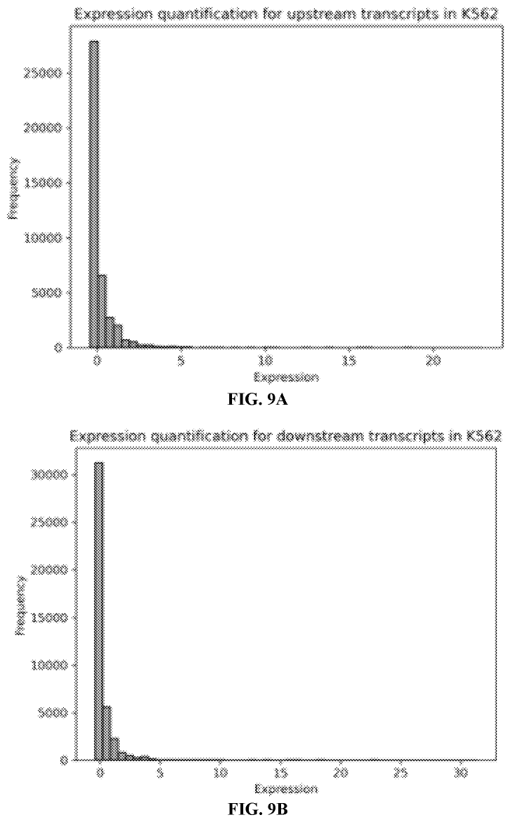

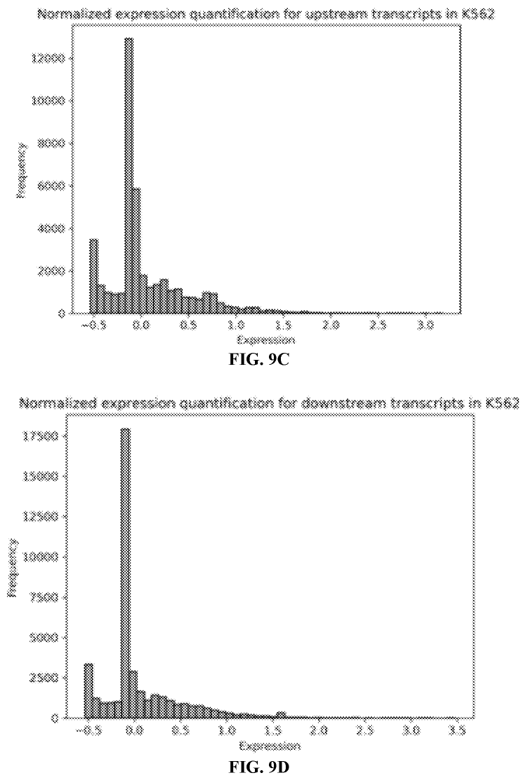

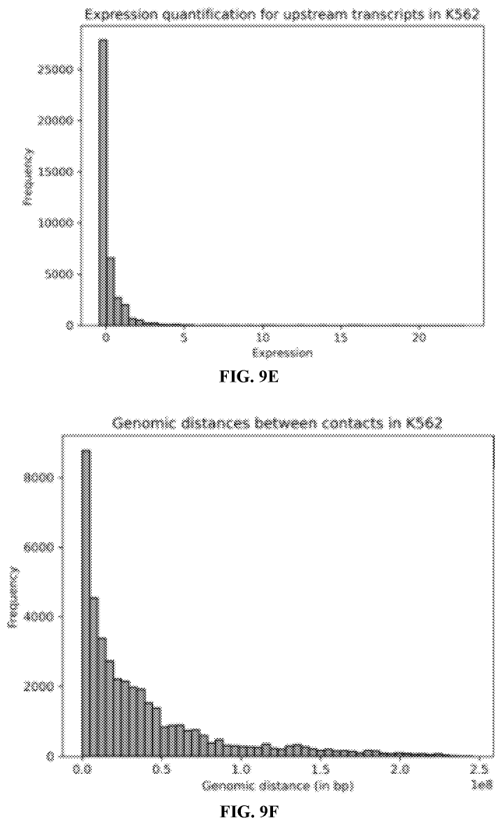

Prior to training a neural network using GReX data, a baseline contact prediction model was trained using eRNA and gene transcript counts quantified via a nuclear run-on assay in the K562 cell line (FIG. 9A-9F). Because nuclear run-on assays are considered a gold standard for detecting nascent eRNA transcription, a model trained on these expression data was intended to serve as a benchmark against which a GReX-based model could be compared.

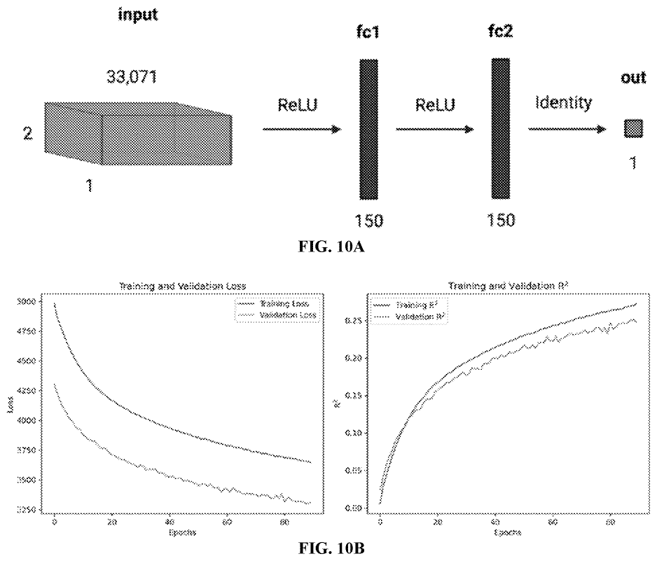

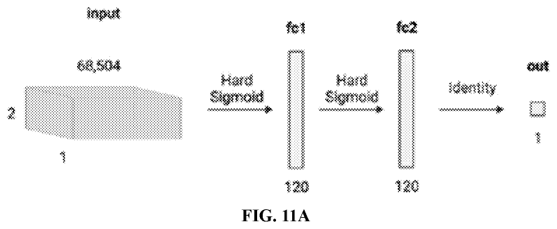

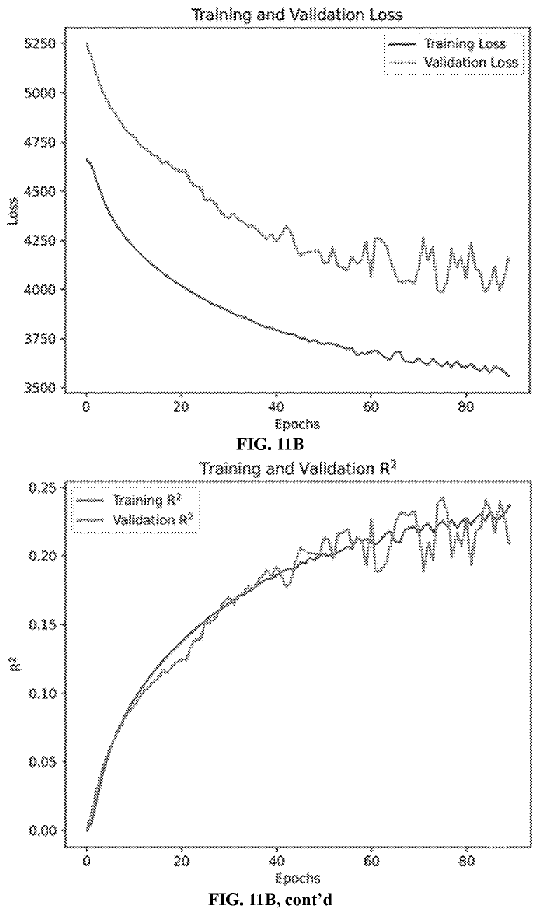

An initial neural network model was trained using eRNA and canonical gene transcription to predict chromatin contacts in the K562 cell line. Hyperparameter tuning was conducted across a predefined search space, and five-fold cross-validation was used to quantify model performance (see Methods). The optimal model architecture (FIG. 10A) consisted of two neurons in the input layer (for the normalized expression levels of the upstream and downstream transcripts), two hidden layers with 150 neurons each (using a ReLU activation function), and a single output neuron. Hidden weights were initialized using a normal distribution, whereas output-layer weights were initialized with zeros. The model was trained for 90 epochs with a batch size of 160. The Adagrad optimizer was used with a learning rate of 0.2. This optimal architecture achieved a mean R² of 0.23 across the validation folds (FIG. 10B) and an R² of 0.27 in the independent test set.

### Example 20: GReX of Enhancer-Gene Pairs Predict Chromatin Contact Frequency

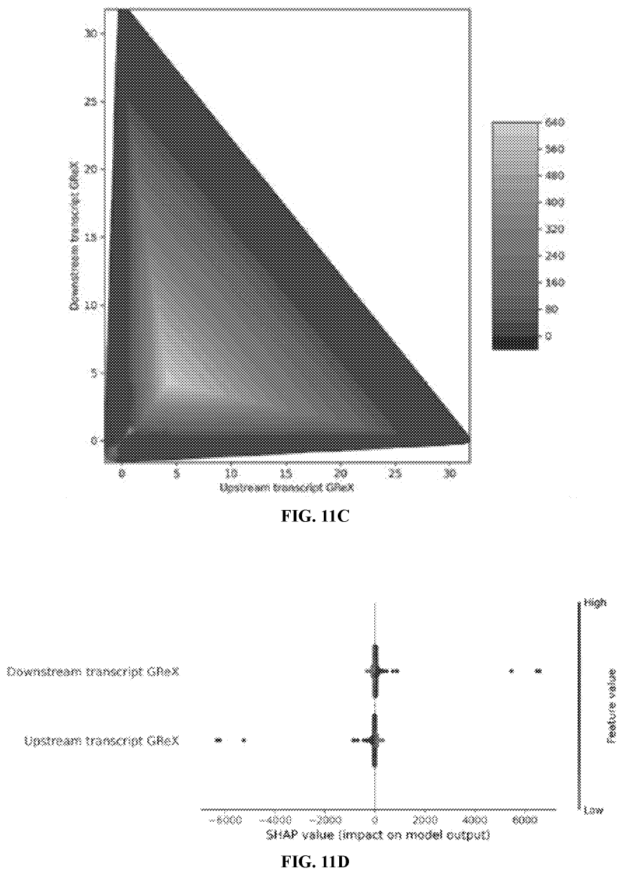

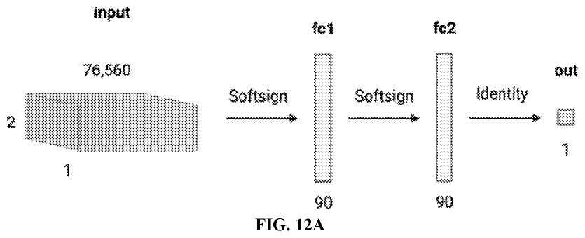

Fully connected neural networks were trained to model the non-linear relationship between GReX and chromatin contact frequency in whole blood and cerebellum. The optimal architecture for the whole blood-based model (FIG. 11A) comprised two neurons in the input layer (for the GReX values of the upstream and downstream transcripts), two hidden layers with 120 neurons each (using a hard sigmoid activation function), and a single output neuron. Hidden-layer weights were initialized using a Kaiming uniform distribution, whereas output-layer weights were initialized using a Kaiming normal distribution. Training was conducted for 90 epochs with a batch size of 110, using the NAdam optimizer with a learning rate of 0.01. A mean R² of 0.22 was achieved in both the validation folds (FIG. 11B) and the independent test set (FIG. 11C). Notably, predictive performance was maintained when the whole blood GReX-based model was evaluated in the K562-derived nuclear run-on dataset (R²=0.15).

Among the 17,126 transcript pairs in the test set, a median relative error E (see Methods) of 2.80 was observed. The best-predicted contact pair in these data was between enhancer ENSR00000041089 and gene PELI3 (true value=6.0, prediction=5.95, E=7.0×10⁻³), a protein-coding gene associated with erythrocyte and lymphocyte counts.

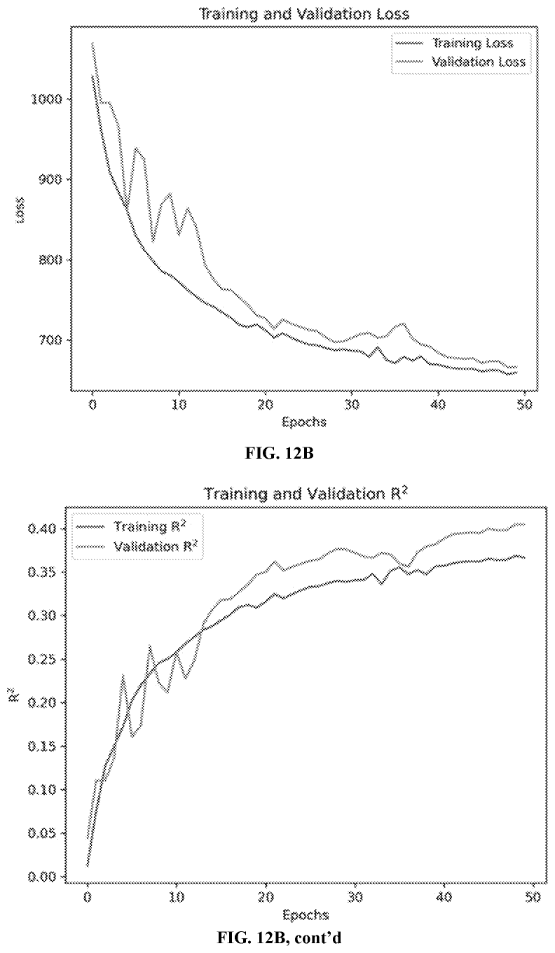

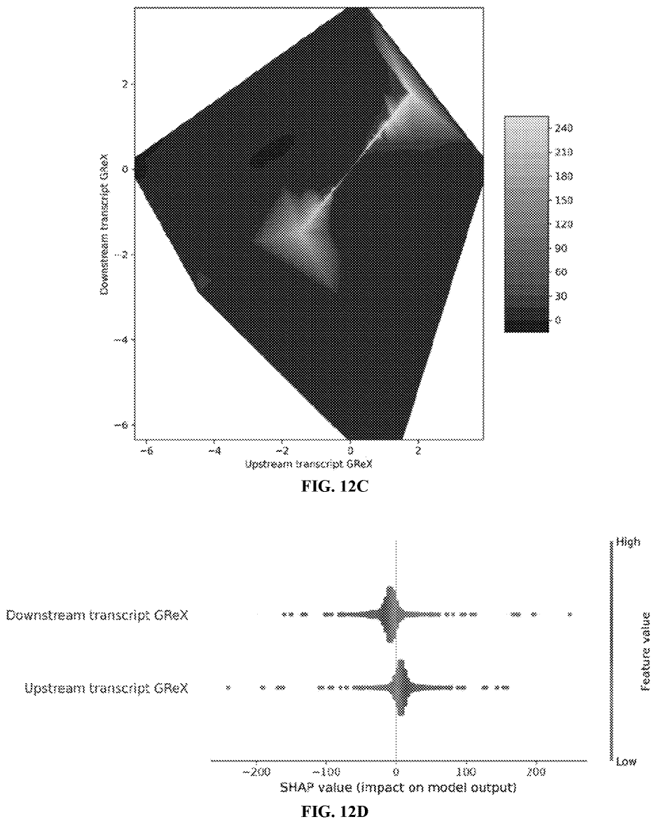

The optimal cerebellum-based model architecture (FIG. 12A) consisted of two hidden layers with 90 neurons each (using the Softsign activation function). Weights in these hidden layers were initialized using a Xavier normal distribution, whereas weights in the output layer were initialized using a uniform distribution. The model was trained for 50 epochs with a batch size of 80, using Adagrad as the optimizer with a learning rate of 0.3. The optimal model achieved a mean R² of 0.37 across the validation folds and 0.38 in the independent test set (FIG. 12B), capturing non-linear patterns of GReX associated with contact frequency (FIG. 12C). The two-dimensional GReX profile for contact pairs was also found to be constrained to a subregion of the total space (FIG. 12C).

In addition to the increased prediction R² in cerebellum, a decreased median relative error was observed in the cerebellum test set compared with whole blood (E=0.85). The best-predicted contact pair among the 19,141 in these data was between enhancer ENSR00000186261 and WDR41 (true value=1.0, prediction=0.99, E=1.80×10⁻⁴), a protein-coding gene implicated in frontotemporal dementia and amyotrophic lateral sclerosis.

Using the same respective test sets for whole blood and cerebellum, SHapley Additive explanations (SHAP) was applied to determine the relative contributions of the upstream and downstream GReX features to model prediction. In both tissues, the mean relative contribution of the downstream transcript was greater than that of the upstream transcript (60.67% versus 39.33% in whole blood and 53.06% versus 46.94% in cerebellum; FIG. 12D).

Different optimal hyperparameters were obtained for the 3D contact frequency model in each tissue, and cross-tissue portability was therefore evaluated. A predictive model achieving good performance in a tissue not available during training would be expected to capture high-level principles underlying the global relationship between expression and chromatin contact rather than stochastic patterns specific to the training tissue.

The whole blood-based model was applied to predict contact frequency in previously unseen cerebellum data. Across the 95,701 unique enhancer-enhancer and enhancer-gene pairs in these cerebellum data, a prediction R² of 0.01 was obtained, representing a marked decrease relative to the R² of 0.22 observed in the tissue-matched test set.

Performance of the cerebellum-trained model was also evaluated on the 85,630 transcript pairs in the whole blood data. Markedly higher cross-tissue performance was observed, with a prediction R² of 0.18 in whole blood, approaching the performance of the whole blood-based model in the same tissue.

### Example 21: Contact Frequency Shows Low Negative Correlation with Genomic Distance

Linear genomic distance between two loci has previously been shown to predict contact frequency. However, unlike genomic distance (which is largely tissue-type independent), three-dimensional chromatin interaction displays highly tissue-specific patterns, particularly when non-ubiquitously expressed genes are involved.

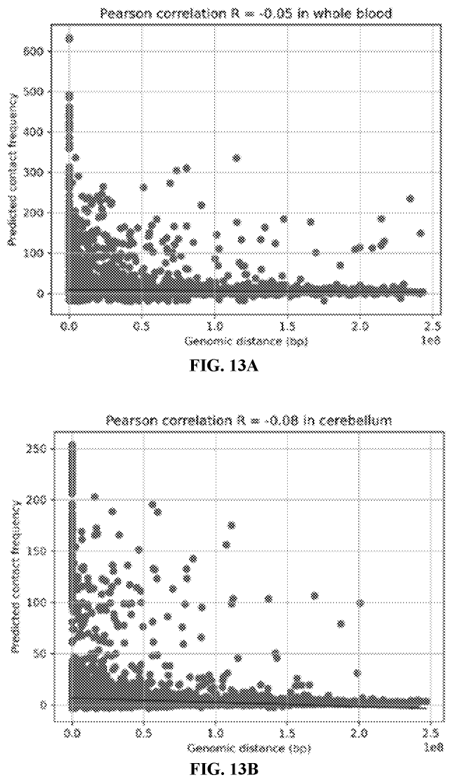

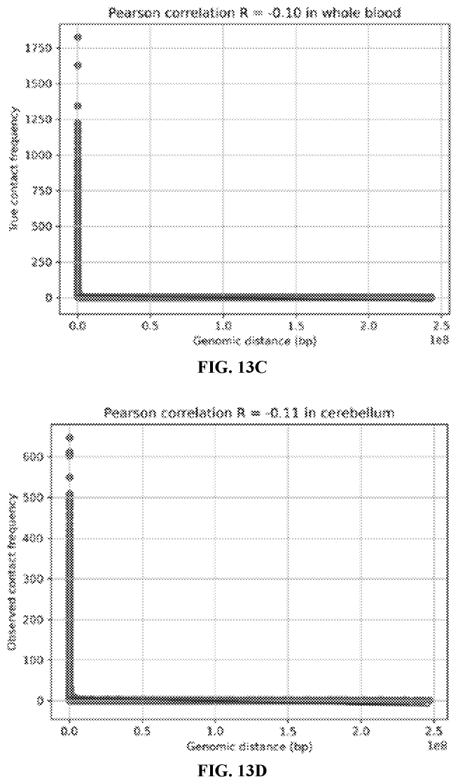

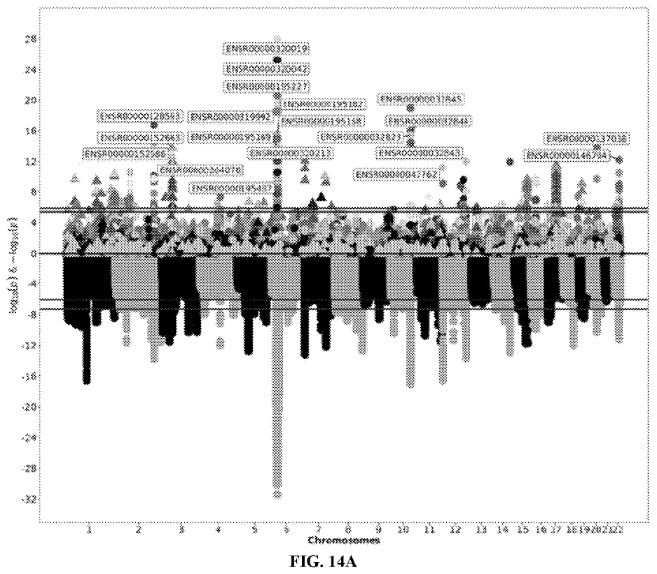

To evaluate the extent to which genomic distance may have influenced model predictions, the Pearson correlation between genomic distance for each transcript pair (as used in the deep learning models' test sets) and both predicted and observed contact frequency was calculated (FIG. 13A-13D). Across the 17,126 transcript pairs in whole blood and the 19,141 transcript pairs in cerebellum, correlations with predicted contact frequency (R=−0.05 and R=−0.08, respectively) and with observed contact frequency (R=−0.10 in whole blood and R=−0.11 in cerebellum) were low, suggesting that deep learning-based contact frequency predictions were largely independent of genomic distance.

### Example 22: TWAS Identifies eRNA and Canonical Gene Associations with SCZ Risk

FUSION-based models of enhancer expression in two brain regions, the dorsolateral prefrontal cortex (DLPFC) and anterior cingulate cortex (ACC), were previously trained in a prior study. Using these models, which included 8,702 unique enhancers, a TWAS of schizophrenia (SCZ) was performed, yielding 98 enhancer associations outside of the major histocompatibility complex (MHC) region. Using eRNA models encompassing 14,471 unique enhancers expressed across 49 cell and tissue types, including more diverse sampling of brain regions, TWAS was performed using the same SCZ GWAS results. The cell and tissues types were Whole Blood, Vagina, Uterus, Thyroid, Testis, Stomach, Spleen, Small Intestine Terminal Ileum, Skin Sun Exposed Lower leg, Skin Not Sun Exposed Suprapubic, Prostate, Pituitary, Pancreas, Ovary, Nerve Tibial, Muscle Skeletal, Minor Salivary Gland, Lung, Liver, Kidney Cortex, Heart Left Ventricle, Heart Atrial Appendage, Esophagus Muscularis, Esophagus Mucosa, Esophagus Gastroesophageal Junction, Colon Transverse, Colon Sigmoid, Cells EBV-transformed lymphocytes, Cells Cultured fibroblasts, Breast Mammary Tissue, Brain Substantia nigra, Brain Spinal cord cervical c-1, Brain Putamen basal ganglia, Brain Nucleus accumbens basal ganglia, Brain Hypothalamus, Brain Hippocampus, Brain Frontal Cortex_BA9, Brain Cortex, Brain Cerebellum, Brain Cerebellar Hemisphere, Brain Caudate basal ganglia, Brain Anterior cingulate cortex BA24, Brain Amygdala, Artery Tibial, Artery Coronary, Artery Aorta, Adrenal Gland, Adipose Visceral Omentum, and Adipose Subcutaneous.

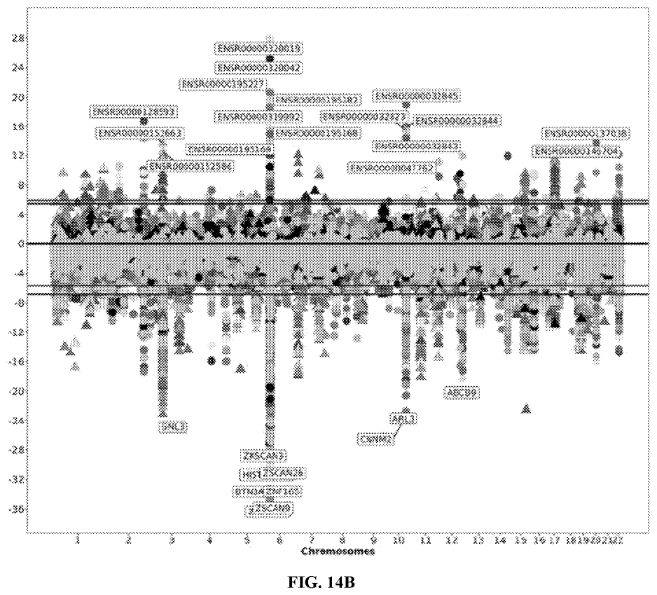

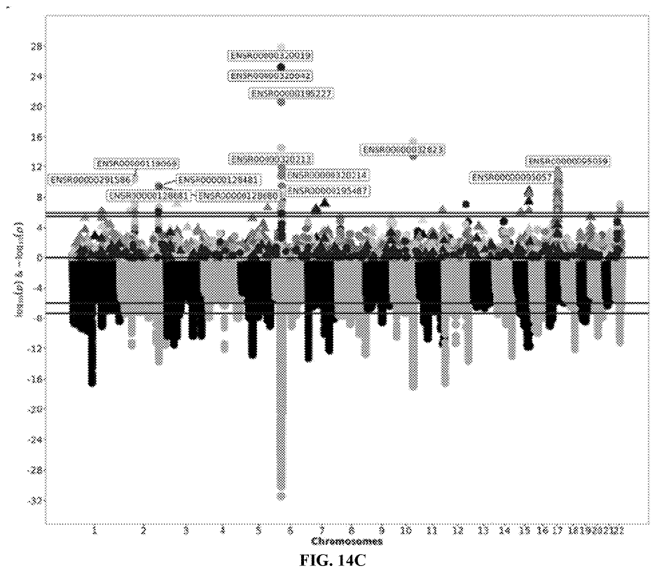

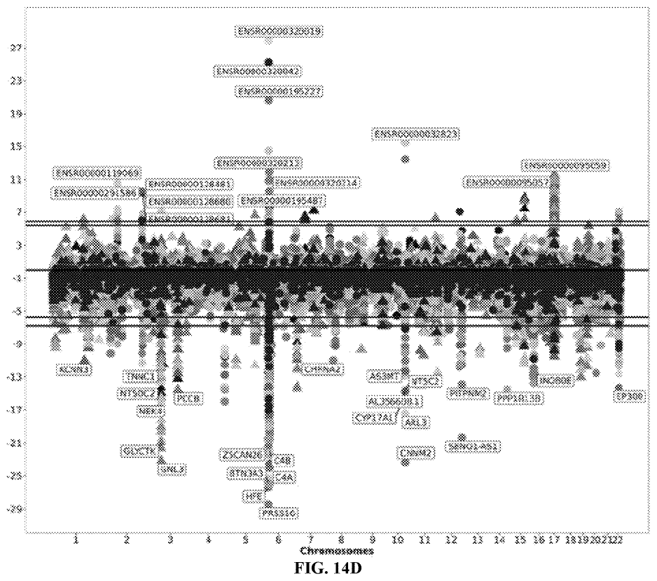

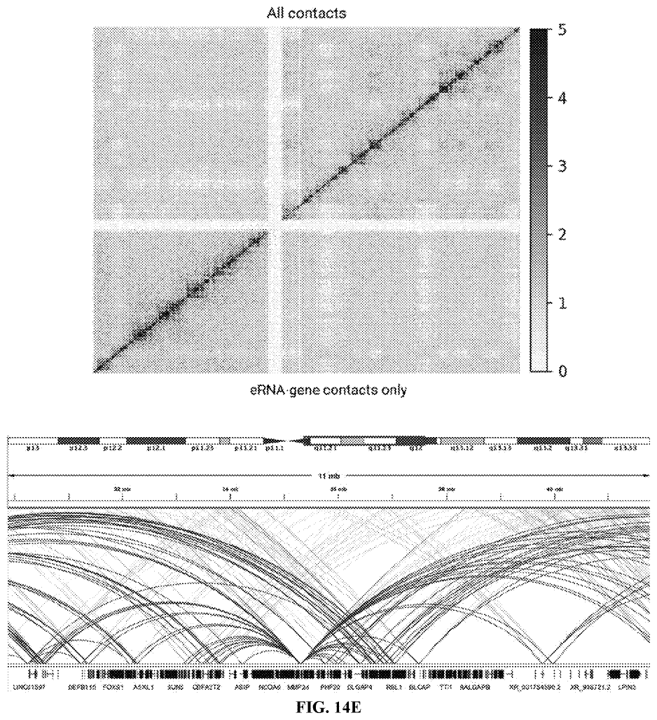

Despite a more stringent multiple-testing threshold for genome-wide significance (p<1.23×10⁻⁶), 392 significant enhancer-tissue associations outside of the MHC region were identified (FIG. 14A-14B), including 114 in the brain (FIG. 14C-14D), representing 133 unique enhancers. Top associations in the brain included ENSR00000320019 (p=1.31×10⁻²⁸ in amygdala), ENSR00000320042 (p=5.94×10⁻²⁶ in substantia nigra), ENSR00000195227 (p=2.48×10⁻²¹ in cortex), ENSR00000032823 (p=3.67×10⁻¹⁶ in cerebellum), and ENSR00000195227 (p=3.24×10⁻¹⁵ in hippocampus).

TWAS was also performed using PrediXcan models of canonical gene expression previously trained in the same cell and tissue types. Relative to FUSION-based gene expression models, which included 10,669 unique genes across two brain regions (204 of which were associated with SCZ), the PrediXcan-based models included 26,133 unique genes expressed across 49 cell and tissue types, including 17,843 genes expressed in one or more brain regions. Across cell and tissue types, 2,755 gene-tissue associations outside of the MHC region reached genome-wide significance (p<1.45×10⁻⁷), including 498 in the brain. Top hits within the brain included PRSS16 (p=3.53×10⁻²⁹ in cerebellar hemisphere and p=5.30×10⁻²⁶ in cerebellum), HFE (p=3.71×10⁻²⁷ in cortex), BTN3A3 (p=1.58×10⁻²⁶ in cortex), CNNM2 (p=4.72× 10⁻²⁴ in frontal cortex BA9), and GNL3 (p=8.15×10⁻²⁴ in cortex), each of which has been previously implicated as an SCZ risk gene.

Whether the original GWAS could have indirectly implicated these TWAS associations was assessed. Of the 206 independent non-MHC loci that reached genome-wide significance in the original GWAS (p<5×10⁻⁸), six loci exhibited only an eRNA association, and 92 loci exhibited only a canonical gene association. An additional 45 loci exhibited both an eRNA and a canonical gene association in at least one tissue type, indicating substantial mirroring between eRNA- and canonical-gene-based TWAS associations (FIG. 4b, d). Collectively, the total number of eRNA TWAS associations in the brain (114) could not be fully accounted for by findings from the original GWAS, indicating the presence of independent eRNA associations with disease.

### Example 23: Mendelian Randomization Illuminates Causal Mechanisms Underlying SCZ Risk

Mendelian randomization (MR) was used to investigate the causality of eRNA and canonical gene associations with SCZ risk. This analysis was conducted for each genome-wide significant association (p<1.23×10⁻⁶ for eRNA associations and p<1.45×10⁻⁷ for canonical gene associations).

Of the 392 significant TWAS eRNA associations outside of the MHC region, 222 (56.63%) showed evidence of a causal effect on SCZ in MR, representing 104 unique transcribed enhancers. Among these putatively causal eRNA effects, 64 were observed in the brain, representing 34 unique enhancers. Of the 2,755 significant gene associations identified in the canonical gene-based TWAS, 1,297 (47.07%) were predicted to be causal, representing 396 unique genes. Among these putatively causal effects, 226 were observed in the brain, representing 130 unique genes.

The presence of putatively causal enhancers and canonical genes within a disease-associated locus raised the question of which transcript type (eRNA or canonical gene) was causally “upstream” in the regulatory network. It was evaluated whether a causal eRNA effect on SCZ risk could be attributed to eRNA expression influencing cis contact with a canonical gene, or whether the eRNA association was not mediated through canonical gene expression. To evaluate physical contact, high-resolution enhancer-gene contact maps captured by Hi-C in astrocytes of the cerebellum were leveraged. In brain, 14 causal (MR-supported) transcribed enhancers were identified as being in physical contact with a causal canonical gene, representing 10 unique enhancer-gene pairs composed of four unique eRNAs and nine unique canonical genes. In addition, 57 causal enhancer-tissue pairs were identified that were not in contact with a causal canonical gene in the Hi-C data, representing 33 unique eRNAs. Finally, 232 causal canonical gene-tissue pairs were identified in brain that were not in contact with a causal eRNA, representing 127 unique genes. The low proportion of physical interaction between causal enhancers and causal canonical genes (approximately 4.6% of all causal transcript-tissue pairs) suggested that eRNA- and gene-related mechanisms in the context of SCZ risk were largely independent.

### Example 24: Exploring the Epigenomic Context of Causal eRNAs Associated with SCZ

Several potential mechanisms of eRNA activity have been described in the literature and can be broadly divided into two classes: contact-dependent and contact-independent mechanisms. Under the contact-dependent model, eRNAs interact with the cohesin complex to promote interactions between the transcribed enhancer and nearby genes. Under a contact-independent model, transcribed eRNAs recruit transcription factors and other chromatin modifiers to influence chromatin state and activity. For example, increased recruitment of transcription factors such as YY1 has been associated with eRNA expression. Interactions with histone acetyltransferases CBP and p300 have also been reported, with associated increases in H3K27ac. In addition, interactions with the PRC2 complex have been described, with inhibition of deposition of the repressive mark H3K27me3. The resulting open chromatin state can facilitate assembly of super-enhancer complexes and/or transcription of nearby genes.

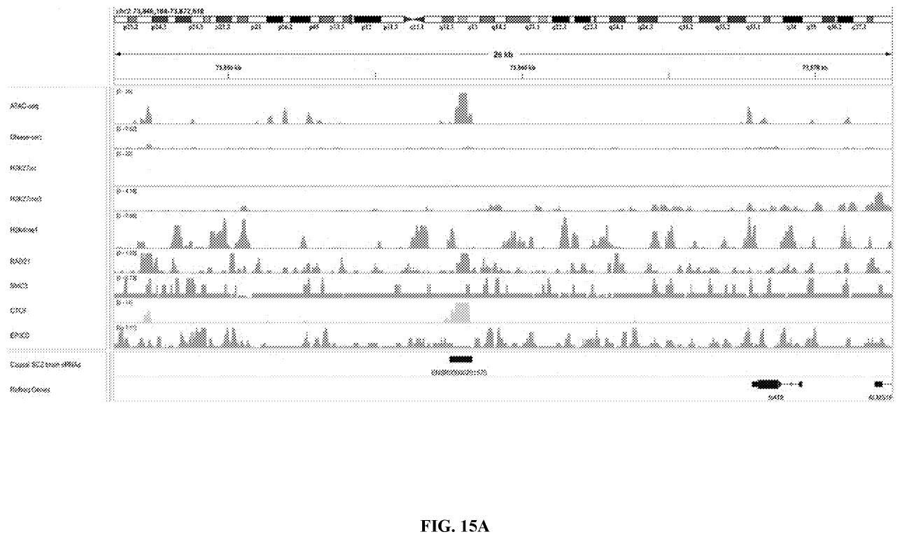

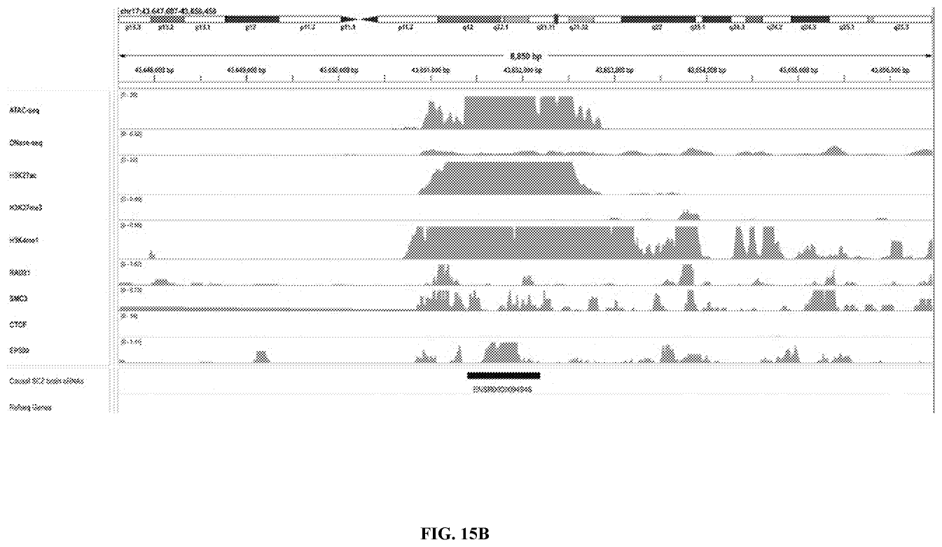

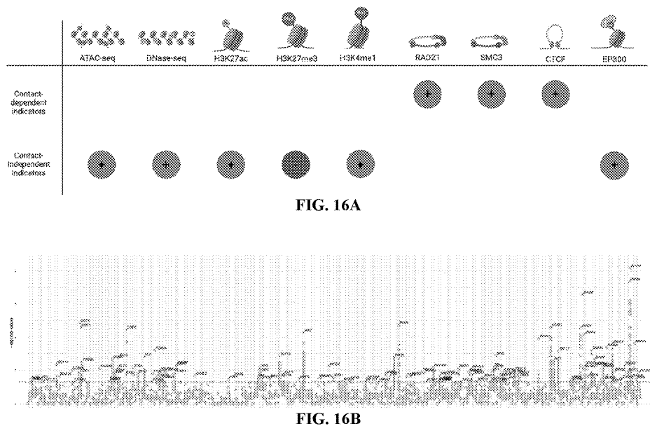

To evaluate whether brain-specific, causal eRNAs might influence SCZ risk via contact-dependent or contact-independent mechanisms, chromatin accessibility profiles (ATAC-seq and DNase-seq) and ChIP-seq profiles of informative histone modifications (H3K27ac, H3K27me3, and H3K4me1) and chromatin-associated proteins (RAD21, SMC3, CTCF, and EP300) in the SK—N—SH neuronal cell line were used to characterize the regulatory landscape of these disease-associated transcripts. If SCZ risk were influenced through direct mediation of enhancer-gene interactions, strong enrichment of cohesin subcomponents (RAD21 and SMC3) or CTCF would be expected. However, among the 34 unique causal eRNAs tested, RAD21 enrichment was observed in only three (~9%), SMC3 enrichment in only one (~3%), and CTCF enrichment in only two (6%) (FIG. 15A). Notably, enrichment of these chromatin contact-associated proteins was not observed within the subset of eRNAs that were in 3D contact with a causal gene. Taken together with the overall low proportion of causal eRNAs in physical contact with causal canonical genes, these results did not support the contact mediation model as a plausible explanation for eRNA associations with SCZ risk.

An alternative model was then evaluated. If causal eRNA expression contributes to maintaining an open chromatin state, enrichment of ATAC-seq and DNase-seq peaks, along with enrichment of EP300 and H3K27ac and depletion of H3K27me3, would be expected. If super-enhancer assembly occurs in these regions, enrichment of H3K4me1 would also be expected. Consistent with these expectations, overlap with ATAC-seq or DNase-seq peaks was observed for 12 (~35%) of the 34 causal eRNA regions, EP300 or H3K27ac enrichment was observed for seven (~21%), and H3K4me1 enrichment was observed for six (~18%) (FIG. 15B). In total, enrichment for at least one open chromatin-associated mark was observed for 25 (~74%) of the 34 causal eRNAs. Notably, H3K27me3 was absent from these same causal enhancer regions. Collectively, these results supported a contact-independent model of eRNA activity, in which causal transcribed enhancers influence SCZ risk through roles in establishing or maintaining an open chromatin state.

### Example 25: Motif Enrichment Analysis within SCZ-Associated eRNAs

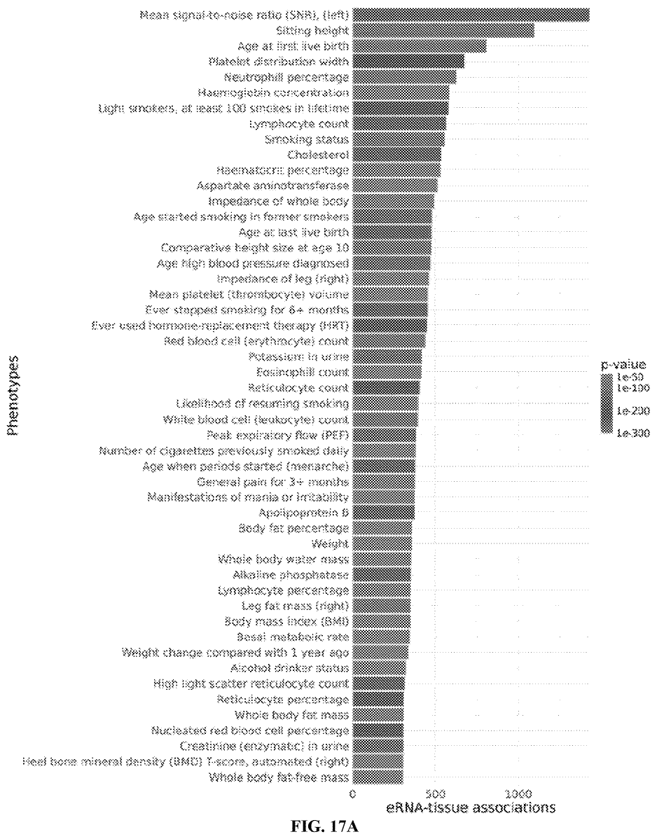

In addition to recruitment of chromatin modifiers, contact-independent eRNAs can “capture” transcription factors (TFs), increasing TF occupancy at the transcribed enhancer. To determine whether SCZ-associated enhancers were enriched for specific transcription factor binding sites, motif enrichment analysis was performed for all putative causal eRNA associations in brain. Across these 34 unique sequences, motif enrichment (FDR<0.05) was observed for 135 TFs (FIG. 16B), several of which (EGR2, SOX10, TCF4/ITF2, and SP4) have previously been implicated in SCZ. Enrichment of binding motifs for these factors at SCZ-associated eRNAs suggests a possible trans mechanism of eRNA-mediated SCZ risk, in which genetically regulated eRNA expression levels influence TF occupancy at disease-relevant enhancers.

### Example 26: Phenome-wide TWAS of eRNAs in the UK Biobank

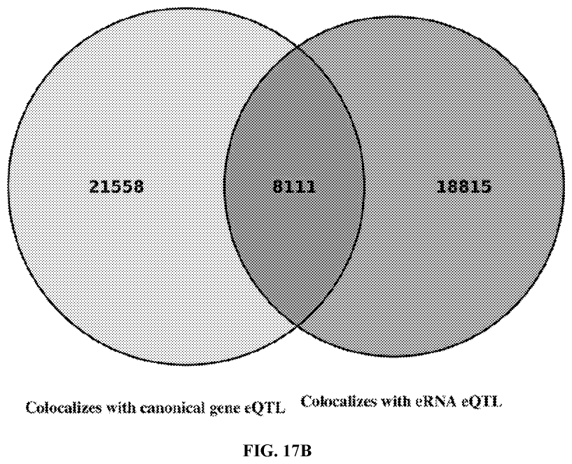

TWAS was performed across 4,671 complex traits using the eRNA models. A total of 467 traits were identified with at least one genome-wide significant (p<2.60×10⁻¹⁰) eRNA association (FIG. 17A), representing 88,348 significant eRNA-tissue associations.

Traits most highly enriched for significant eRNA associations included mean signal-to-noise ratio, a measure of hearing ability (1,430 significant eRNA-tissue associations; lead association p=6.49×10⁻¹⁴³), as well as sitting height, a well-studied phenotype known to be highly polygenic (1,094 significant eRNA-tissue associations; lead association p=1.71× 10⁻¹⁰⁶). Additional phenotypes highly enriched for significant eRNA-tissue associations included blood cell traits such as platelet distribution width (676 associations), neutrophil percentage (625 associations), hemoglobin concentration (583 associations), lymphocyte count (562 associations), and leukocyte count (394 associations); as well as smoking status (554 associations), cholesterol (536 associations), manifestations of mania or irritability (374 associations), weight (357 associations), and skin color (221 associations). TWAS summary statistics are presented as a comprehensive tissue-wide resource of eRNA associations across the human phenome.

### Example 27: Enhancer Perturbation Links eRNAs to Canonical Target Gene Expression

Epigenomic analysis of causal SCZ-associated eRNAs supported a context-independent, rather than contact-dependent, model. However, these data alone cannot fully explain the mechanisms by which eRNA expression influences disease risk. To investigate whether disease-associated eRNAs affect canonical gene expression, CRISPR perturbation assays in the K562 cell line targeting 109 complex trait-associated, transcribed enhancers identified by TWAS were used. Among these 109 enhancers, 14 (12.84%) were identified for which CRISPR perturbation resulted in a significant change in expression of a corresponding gene. Notably, Hi-C contacts were not observed between any of these 14 eRNAs and their target gene(s). eQTL mapping in whole blood was also performed for both eRNAs and canonical genes. Among the 22 unique eRNA-gene pairs identified using CRISPR perturbation, only one mapped SNP eQTL (FDR<0.1) was observed to be shared by both the eRNA and canonical gene in a pair. Collectively, these results suggested that these eRNAs mediated canonical gene expression independently of chromatin contacts or shared SNP eQTLs.

Among disease-relevant eRNAs linked to canonical gene expression were ENSR00000013481 and ENSR00000032851 (both associated with SCZ), ENSR00000117322 (associated with manifestations of mania or irritability), and ENSR00000320642 (associated with hypertension). Perturbation of SCZ-associated eRNAs ENSR00000013481 and ENSR00000032851 was associated with decreased expression of VPS45 (fold change=0.75, p=1.21×10⁻³) and NT5C2 (fold change=0.74, p=7.66×10⁻⁴), respectively. Both genes have previously been implicated in SCZ and reached significance in the canonical gene-based TWAS of SCZ.

In addition to the two SCZ-associated eRNAs, perturbation of ENSR00000117322 (associated with manifestations of mania or irritability) was associated with decreased expression of the SCZ-associated gene RTN4 (fold change=0.83, p=6.91×10⁻⁹), while perturbation of hypertension-associated eRNA ENSR00000320642 was associated with decreased MRPS10 expression (fold change=0.82, p=1.42×10⁻⁸). This gene encodes a mitochondrial ribosomal protein and has previously been linked to cardiac disorders. Neither of these eRNA-linked genes reached significance in a canonical gene-based TWAS.

### Example 28: Inclusion of eRNAs Increases GWAS Signals Explained by an eQTL

Systematic differences between genetic effects identified for complex traits through GWAS and genetic effects identified for gene expression through eQTL mapping have been reported, such that most GWAS signals are not explained by known eQTLs. Several strategies have been proposed to bridge this colocalization gap, including the use of larger sample sizes to increase eQTL mapping power, mapping eQTLs across more expansive sets of cell and tissue types, and considering QTLs that modulate chromatin structure and splicing. Whether incorporation of eRNA eQTLs could improve interpretability of GWAS signals was assessed.

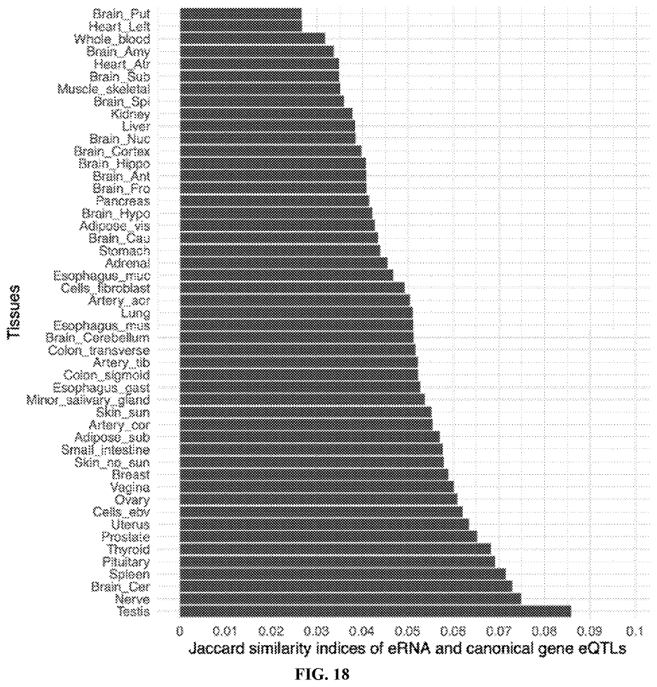

Across all tissues, a mean Jaccard similarity index of 0.05 was observed between eRNA eQTLs (Methods) and canonical gene eQTLs, indicating low overlap between the two eQTL subclasses (FIG. 18). The highest overlap was observed in testis (Jaccard similarity index=0.09), whereas the lowest overlap was observed in brain putamen (Jaccard similarity index=0.03).

Previously reported discordance between GWAS signals and canonical gene eQTLs has been attributed in part to the tendency for eQTLs to be proximal to genes, whereas GWAS signals are more distal. In contrast to canonical gene eQTLs, eRNA-specific eQTLs were found to be significantly more distal to a transcription start site (TSS), consistent with the distribution of GWAS hits (median distance 41.93 kb versus 14.36 kb; Mann-Whitney U test p<2.2×10⁻¹⁶).

Whether inclusion of eRNA eQTLs could help bridge the gap between eQTLs and GWAS signals was evaluated through colocalization analysis across 996,391 independent, genome-wide significant (p<5×10⁻⁸) associations from the UK Biobank, representing 4,671 complex traits. A total of 26,926 GWAS associations were identified as colocalizing with an eRNA eQTL (posterior probability >0.7) in at least one tissue, and an additional 29,669 GWAS associations were identified as colocalizing with a canonical gene eQTL. In total, 48,484 GWAS associations colocalized with either an eRNA or canonical gene eQTL, with 8,111 (16.75%) colocalizing with both and 40,303 (83.25%) colocalizing with only one eQTL subclass. Notably, 18,815 GWAS signals colocalized exclusively with an eRNA eQTL, corresponding to a 63% increase in the total number of GWAS signals with shared causal variants when eRNA eQTLs were considered (FIG. 17B). Collectively, these results indicate that inclusion of eRNA eQTLs can substantially improve mechanistic interpretation of GWAS associations.

### Discussion of the Examples

In silico models of genetically regulated eRNA expression were developed, yielding several notable findings spanning chromatin contact frequency prediction and eRNA-based TWAS.

Genetic Control of eRNA Expression (eQTL Burden)

Across all tissues, eRNA expression exhibited a significantly higher mean number of SNP eQTLs per transcript compared with canonical gene expression. Two possible explanations may account for this observation. Because enhancers exhibit a high degree of tissue-specific and context-dependent activity, eRNA expression may likewise be subject to a higher degree of fine-tuned genetic control than protein-coding genes. Alternatively, the elevated number of SNP eQTLs per eRNA may reflect the rapid evolution of enhancer sequences relative to canonical genes. As non-coding genomic elements, enhancers may tolerate mutation to a greater extent than typical protein-coding genes.

### Imputation at Biobank Scale and Chromatin Contact Frequency Prediction

Genetically determined eRNA expression and canonical gene expression were imputed across more than 70,000 individuals in the large-scale BioVU biobank. Mean genetically regulated expression values generated for two tissues, whole blood and cerebellum, were subsequently used to train two independent neural networks to predict three-dimensional chromatin contact frequency. Previous studies have demonstrated that genomic distance, Hi-C data, and other one-dimensional epigenomic datasets can be used to predict chromatin contact. The present findings demonstrate that genetically regulated expression is likewise predictive of chromatin contact frequency.

### Comparative Performance of Deep Learning Models

Deep learning models trained in cerebellum and whole blood predicted tissue-dependent contact frequency with substantially greater performance than corresponding linear and non-linear regression models, which exhibited limited predictive power. Notably, the whole blood-based genetically regulated expression model retained comparable predictive accuracy in a genome-wide nuclear run-on dataset from K562 cells, indicating that key patterns underlying the relationship between expression and contact frequency were captured.

### Cross-Tissue Portability and Potential Explanations

While the cerebellum-based deep learning model appeared portable to a tissue not observed during training (R²=0.18 in whole blood), the whole blood-based model demonstrated limited predictive power in cerebellum (R²=0.01). Two factors may contribute to this discrepancy in cross-tissue performance. First, whole blood is highly heterogeneous relative to other tissues, comprising a variety of cell types and metabolites whose relative proportions can vary substantially within a given sample. Second, gene expression in whole blood is highly dynamic, with rapid changes induced by diverse environmental stimuli, including seasonal variation. Reduced stochasticity of cerebellar expression may partially explain the greater cross-tissue generalizability of the cerebellum-based model, which may capture more fundamental patterns governing the relationship between genetically regulated expression and chromatin contact frequency.

### Functional Interpretation at Scale and Contact Map Challenges

Incorporation of functional information enhances the interpretability of the phenotypic consequences of complex genetic variation. However, generation of rich omics datasets at biobank scale are often infeasible due to cost, time, and limited sample availability. In silico approaches such as PrediXcan have provided a means of overcoming this limitation by enabling imputation of genetically regulated expression at the gene level and for individual splice isoforms using germline variation alone.

Construction of high-resolution, tissue-specific chromatin contact maps remains methodologically challenging due to the quadratic scaling of the data. In this context, genetically regulated expression models were used to gain further insight into three-dimensional chromatin organization by coupling genetically determined transcription with contact map prediction.

eRNA-Based TWAS and Phenome-Wide Resource Generation

Because the majority of genetic associations with complex disease reside in non-coding regions of the genome, transcriptome-wide association studies have become a valuable approach for inferring functionally relevant genes within GWAS loci. For example, prior TWAS of schizophrenia identified non-coding variation at locus 16p11.2 associated with increased expression of MAPK3. The mechanistic contribution of eRNAs to disease risk remains incompletely understood. Recent studies of neuropsychiatric phenotypes have demonstrated that enhancer expression quantitative trait loci, or SNPs regulating enhancer transcription in the brain, can be leveraged to investigate disease mechanisms. These studies concluded that much of the disease heritability captured by eRNAs is independent of that explained by eQTLs for protein-coding genes. Based on these findings, eRNA expression in non-neuronal tissues is likely to represent an important contributor to disease biology across the human phenome. Accordingly, eRNA-based models trained across a large collection of tissues have been made available to facilitate downstream genomic analyses.

Using eRNA-based TWAS, 392 schizophrenia-associated eRNAs were identified, corresponding to 104 unique eRNAs. This analytical framework was subsequently applied to a set of phenome-wide GWAS traits in the UK Biobank, yielding a curated resource for investigating the phenotypic consequences of enhancer activity.

Perturbation Evidence and Genetic Independence of eRNA and Canonical Gene Regulation

Among a sample of 109 complex trait-associated eRNAs, experimental perturbation resulted in altered canonical gene expression in approximately 12% of cases. Because these CRISPR perturbation data were derived from the leukemic K562 cell line, regulatory effects may be more pronounced in other cell types. Notably, among eRNAs linked to canonical genes in K562 cells, no evidence of physical interaction was observed in Hi-C contact data. In addition, no sharing of eQTLs between eRNAs and canonical genes was detected across linked pairs. These findings suggest that eRNA and canonical gene expression are regulated by largely independent genetic mechanisms, rather than by a pleiotropic model in which shared eQTLs jointly regulate both transcripts at a locus.

### Epigenomic Context and Assay Considerations

Given the low frequency of observed contacts between complex trait-associated eRNAs and canonical genes, the epigenomic context of these enhancer sequences was examined to further elucidate potential mechanisms underlying disease risk. Among schizophrenia-associated causal eRNAs, strong enrichment was observed for features associated with open chromatin, accompanied by relatively weak enrichment for features associated with chromatin loop formation. A key limitation of these analyses is that the ChIP-seq assays employed-particularly those profiling EP300, RAD21, SMC3, and CTCF-measure protein binding to DNA rather than RNA. Due to the lack of available RIP-seq and CLIP-seq datasets for these factors in neuronal cells, direct assessment of eRNA-protein interactions were not possible, necessitating reliance on DNA binding as a proxy. More comprehensive characterization of the epigenomic consequences of eRNA perturbation remains an important direction for future research.

### Mechanistic Interpretation: Contact-Dependent Regulatory Model

Nevertheless, the functional evidence presented here, together with significant transcription factor binding motif enrichment within schizophrenia-associated causal eRNAs, supports a contact-dependent regulatory model. Under this model, genetically regulated eRNA expression influences recruitment of chromatin modifiers and transcription factor occupancy at corresponding enhancers. Genetic regulation of these contact-dependent mechanisms would be expected to influence downstream canonical gene expression, consistent with observed effects following CRISPR perturbation of selected disease-associated enhancers.

Increased GWAS Signal Explanation by Incorporating eRNAs (Colocalization Gap Reduction)

Analysis of the comprehensive catalog of disease-associated eRNAs in the UK Biobank demonstrates that inclusion of eRNAs substantially reduces the colocalization gap with GWAS traits. Specifically, a 63% increase was observed in the number of significant GWAS associations explainable by eQTLs when eRNAs were incorporated, relative to analyses restricted to canonical genes alone.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Abdennur, N. & Mirny, L. A. Cooler: scalable storage for Hi-C data and other genomically labeled arrays. Bioinformatics 36, 311-316 (2020).
2. Abugessaisa, I. et al. FANTOM5 CAGE profiles of human and mouse reprocessed for GRCh38 and GRCm38 genome assemblies. Sci. Data 4, 170107 (2017).
3. Abugessaisa, I. et al. refTSS: A Reference Data Set for Human and Mouse Transcription Start Sites. J. Mol. Biol. 431, 2407-2422 (2019).
4. Agarwal, A. & Chen, L. DeepPHIC: predicting promoter-centered chromatin interactions using a novel deep learning approach. Bioinformatics 39, (2023).
5. Altmãe, S., Molina, N. M. & Sola-Leyva, A. Omission of non-poly(A) viral transcripts from the tissue level atlas of the healthy human virome. BMC Biol. vol. 18, 179 (2020).
6. Amick, J., Tharkeshwar, A. K., Amaya, C. & Ferguson, S. M. WDR41 supports lysosomal response to changes in amino acid availability. Mol. Biol. Cell 29, 2213-2227 (2018).
7. Arnold, P. R., Wells, A. D. & Li, X. C. Diversity and emerging roles of enhancer rna in regulation of gene expression and cell fate. Front Cell Dev. Biol. 7, 377 (2019).
8. Barbeira, A. N. et al. Exploring the phenotypic consequences of tissue specific gene expression variation inferred from GWAS summary statistics. Nat. Commun. 9, 1825 (2018).
9. Betti, M. BioRender.com/s62i616 (2025).
10. Betti, M. BioRender.com/x12w509 (2025).
11. Betti, M. J. & Gamazon, E. eRNA GReX. Zenodo doi.org/10.5281/zenodo. 14027849 (2024).
12. Betti, MJ & Gamazon, E. mjbetti/erna-grex: v1. Zenodo doi.org/10.5281/zenodo.14557414 (2024).
13. Bhattacharya, A. et al. Isoform-level transcriptome-wide association uncovers extensive novel genetic risk mechanisms for neu-ropsychiatric disorders in the human brain. bioRxiv doi. org/10.1101/2022.08.23.22279134 (2022).
14. Bose, D. A. et al. RNA binding to CBP stimulates histone acetylation and transcription. Cell 168, 135-149.e22 (2017).
15. Buxton, D. S., Batten, D. J., Crofts, J. J. & Chuzhanova, N. Predicting novel genomic regions linked to genetic disorders using GWAS and chromosome conformation data—a case study of schizophrenia. Sci. Rep. 9, 17940 (2019).
16. Cao, R. et al. Role of histone H3 lysine 27 methylation in Polycomb-group silencing. Science 298, 1039-1043 (2002).
17. Chen, F. et al. Multi-ancestry transcriptome-wide association analyses yield insights into tobacco use biology and drug repurposing. Nat. Genet. 55, 291-300 (2023).
18. Chen, J. et al. Role played by the SP4 gene in schizophrenia and major depressive disorder in the Han Chinese population. Br. J. Psychiatry 208, 441-445 (2016).
19. Chen, M.-H. et al. Trans-ethnic and Ancestry-Specific Blood-Cell Genetics in 746,667 Individuals from 5 Global Populations. Cell 182, 1198-1213.e14 (2020).
20. Chen, P. et al. Polymorphism of Transferrin Gene Impacts the Mediating Effects of Psychotic Symptoms on the Relationship between Oxidative Stress and Cognition in Patients with Chronic Schizophrenia. Antioxidants (Basel) 11, (2022).
21. Chun, S. et al. Limited statistical evidence for shared genetic effects of eQTLs and autoimmune-disease-associated loci in three major immune-cell types. Nat. Genet. 49, 600-605 (2017).
22. Churchhouse, C. Rapid GWAS of thousands of phenotypes for 337,000 samples in the UK Biobank. Neale lab www.nealelab.is/blog/2017/7/19/rapid-gwas-of-thousands-of-phenotypes-for-337000-samples-in-the-uk-biobank (2017).
23. Connally, N. J. et al. The missing link between genetic association and regulatory function. Elife 11, (2022).
24. Consortium, G. T. Ex The GTEx Consortium atlas of genetic regulatory effects across human tissues. Science 369, 1318-1330 (2020).
25. De Jong, S. et al. Seasonal changes in gene expression represent cell-type composition in whole blood. Hum. Mol. Genet. 23, 2721-2728 (2014).
26. Dekker, J. et al. The 4D nucleome project. Nature 549, 219-226 (2017).
27. Dong, P. et al. Population-level variation in enhancer expression identifies disease mechanisms in the human brain. Nat. Genet. 54, 1493-1503 (2022).
28. Dozat, T. Incorporating Nesterov Momentum into Adam. Proceedings of the 4th International Conference on Learning Representations (2016).
29. Dsouza, K. B. et al. Learning representations of chromatin contacts using a recurrent neural network identifies genomic drivers of conformation. Nat. Commun. 13, 3704 (2022).
30. Duarte, R. R. R. et al. The psychiatric risk gene NT5C2 regulates adenosine monophosphate-activated protein kinase signaling and protein translation in human neural progenitor cells. Biol. Psychiatry 86, 120-130 (2019).
31. Duchi, J., Hazan, E. & Singer, Y. Adaptive Subgradient Methods for Online Learning and Stochastic Optimization. J. Mach. Learn. Res. (2011).
32. ENCODE Project Consortium. An integrated encyclopedia of DNA elements in the human genome. Nature 489, 57-74 (2012).
33. ENCODE Project Consortium et al. Expanded encyclopaedias of DNA elements in the human and mouse genomes. Nature 583, 699-710 (2020).
34. Frankish, A. et al. GENCODE 2021. Nucleic Acids Res 49, D916-D923 (2021).
35. Gamazon, E. & Zhou, D. JTI Version 1.0. Zenodo doi.org/10.5281/zenodo.3842289 (2020).

36 Gamazon, E. R. et al. A gene-based association method for map-ping traits using reference transcriptome data. Nat. Genet. 47, 1091-1098 (2015).
37. Gao, F. et al. A defect in mitochondrial protein translation influences mitonuclear communication in the heart. Nat. Commun. 14, 1595 (2023).

38 Gasperini, M. et al. A genome-wide framework for mapping gene regulation via cellular genetic screens. Cell 176, 377-390.e19 (2019).

39 Gay, N. R. et al. Impact of admixture and ancestry on eQTL analysis and GWAS colocalization in GTEx. Genome Biol. 21, 233 (2020).
40. Ge, T., Chen, C.-Y., Neale, B. M., Sabuncu, M. R. & Smoller, J. W. Phenome-wide heritability analysis of the UK Biobank. PLOS Genet 13, e1006711 (2017).
41. Giambartolomei, C. et al. Bayesian test for colocalisation between pairs of genetic association studies using summary statistics. PLOS Genet 10, e1004383 (2014).
42. Giral, H., Landmesser, U. & Kratzer, A. Into the Wild: GWAS Exploration of Non-coding RNAs. Front Cardiovasc Med 5, 181 (2018).
43. Girgenti, M. J., LoTurco, J. J. & Maher, B. J. ZNF804a regulates expression of the schizophrenia-associated genes PRSS16, COMT, PDE4B, and DRD2. PLOS One 7, e32404 (2012).
44. Gosselin, D. et al. Environment drives selection and function of enhancers controlling tissue-specific macrophage identities. Cell 159, 1327-1340 (2014).
45. Grant, C. E., Bailey, T. L. & Noble, W. S. FIMO: scanning for occurrences of a given motif. Bioinformatics 27, 1017-1018 (2011).
46. GTEx Consortium. et al. Genetic effects on gene expression across human tissues. Nature 550, 204-213 (2017).
47. Gusev, A. et al. Integrative approaches for large-scale transcriptome-wide association studies. Nat. Genet. 48, 245-252 (2016).
48. Gusev, A. et al. Transcriptome-wide association study of schizophrenia and chromatin activity yields mechanistic disease insights. Nat. Genet. 50, 538-548 (2018).
49. Hahn, S. & Kim, D. Physical origin of the contact frequency in chromosome conformation capture data. Biophys. J. 105, 1786-1795 (2013).
50. Han, Z. & Li, W. Enhancer RNA: What we know and what we can achieve. Cell Prolif. 55, e13202 (2022).
51. Harrow, J. et al. GENCODE: the reference human genome annotation for The ENCODE Project. Genome Res 22, 1760-1774 (2012).
52. Hederih, J. et al. Genetic underpinnings of schizophrenia-related electroencephalographical intermediate phenotypes: A systematic review and meta-analysis. Prog. Neuropsychopharmacol. Biol. Psychiatry 104, 110001 (2021).

53 Hervoso, J. L. et al. Splicing-specific transcriptome-wide association uncovers genetic mechanisms for schizophrenia. Am. J. Hum. Genet. 111, 1573-1587 (2024).
54. Hinrichs, A. S. et al. The UCSC Genome Browser Database: update 2006. Nucleic Acids Res 34, D590-8 (2006).
55. Hnisz, D. et al. Super-enhancers in the control of cell identity and disease. Cell 155, 934-947 (2013).

56 Howe, K. L. et al. Ensembl 2021. Nucleic Acids Res 49, D884-D891 (2021).

57 Hu, T.-M., Chen, C.-H., Chuang, Y.-A., Hsu, S.-H. & Cheng, M.-C. Resequencing of early growth response 2 (EGR2) gene revealed a recurrent patient-specific mutation in schizophrenia. Psychiatry Res 228, 958-960 (2015).
58. Huang, J. et al. Dynamic control of enhancer repertoires drives lineage and stage-specific transcription during hematopoiesis. Dev. Cell 36, 9-23 (2016).
59. Imakaev, M. et al. Iterative correction of Hi-C data reveals hallmarks of chromosome organization. Nat. Methods 9, 999-1003 (2012).
60. Iwamoto, K. et al. DNA methylation status of SOX10 correlates with its downregulation and oligodendrocyte dysfunction in schizophrenia. J. Neurosci. 25, 5376-5381 (2005).
61. Kleiveland, C. R. Peripheral Blood Mononuclear Cells. (Springer, 2015).

62 Ko, J. Y., Oh, S. & Yoo, K. H. Functional enhancers as master regulators of tissue-specific gene regulation and cancer development. Mol. Cells 40, 169-177 (2017).
63. Koch, F. et al. Transcription initiation platforms and GTF recruitment at tissue-specific enhancers and promoters. Nat. Struct. Mol. Biol. 18, 956-963 (2011).
64. Koh, E. T., Chi, M. S. & Lowenstein, F. W. Comparison of selected blood components by race, sex, and age. Am. J. Clin. Nutr. 33, 1828-1835 (1980).
65. Kresovich, J. K., Parks, C. G., Sandler, D. P., Weinberg, C. R. & Taylor, J. A. The Role of Blood Cell Composition in Epidemiologic Studies of Telomeres. Epidemiology 31, e34-e36 (2020).
66. Kulakovskiy, I. V. et al. HOCOMOCO: towards a complete collection of transcription factor binding models for human and mouse via large-scale ChIP-Seq analysis. Nucleic Acids Res 46, D252-D259 (2018).
67. Lanfear, D. E., Yang, J. J., Mishra, S. & Sabbah, H. N. Genome-wide approach to identify novel candidate genes for beta blocker response in heart failure using an experimental model. Discov. Med. 11, 359-366 (2011).
68. Li, Q. et al. Enhancer RNAs: mechanisms in transcriptional regulation and functions in diseases. Cell Commun. Signal. 21, 191 (2023).
69. Li, W. et al. Functional roles of enhancer RNAs for oestrogen-dependent transcriptional activation. Nature 498, 516-520 (2013).
70. Li, W., Wong, W. H. & Jiang, R. DeepTACT: predicting 3D chromatin contacts via bootstrapping deep learning. Nucleic Acids Res 47, e60 (2019).
71. Liu, Y. et al. Gene Expression and RNA Splicing Imputation Identifies Novel Candidate Genes Associated with Osteoporosis. J. Clin. Endocrinol. Metab. 105, e4742-57 (2020).
72. Lundberg, S. & Lee, S.-I. A Unified Approach to Interpreting Model Predictions. arXiv [cs.AI] (2017).
73. Marballi, K. K. & Gallitano, A. L. Immediate early genes anchor a biological pathway of proteins required for memory formation, long-term depression and risk for schizophrenia. Front. Behav. Neurosci. 12, 23 (2018).
74. Melka, M. G. et al. Genome-wide scan for loci of adolescent obesity and their relationship with blood pressure. J. Clin. Endocrinol. Metab. 97, E145-50 (2012).
75. Mostafavi, H., Spence, J. P., Naqvi, S. & Pritchard, J. K. Systematic differences in discovery of genetic effects on gene expression and complex traits. Nat. Genet. 55, 1866-1875 (2023).
76. Napoli, S., Munz, N., Guidetti, F. & Bertoni, F. Enhancer RNAs (eRNAs) in cancer: The jacks of all trades. Cancers (Basel) 14, 1978 (2022).
77. Natoli, G. & Andrau, J.-C. Noncoding transcription at enhancers: general principles and functional models. Annu. Rev. Genet. 46, 1-19 (2012).
78. Neale, B. UK Biobank GWAS Round 2. Neale Lab www.nealelab.is/uk-biobank/(2018).
79. Nickolls, J., Buck, I., Garland, M. & Skadron, K. Scalable Parallel Programming with CUDA: Is CUDA the parallel programming model that application developers have been waiting for? Queueing Syst. 6, 40-53 (2008).
80. Panigrahi, A. & O'Malley, B. W. Mechanisms of enhancer action: the known and the unknown. Genome Biol. 22, 108 (2021).
81. Paszke, A. et al. PyTorch: An Imperative Style, High-Performance Deep Learning Library. arXiv [cs.LG] (2019).
82. Pedregosa, F. et al. Scikit-learn: Machine Learning in Python. arXiv [cs.LG] (2012).
83. Pezone, A. et al. RNA stabilizes transcription-dependent chromatin loops induced by nuclear hormones. Sci. Rep. 9, 3925 (2019).
84. Plooster, M., Brennwald, P. & Gupton, S. L. Endosomal trafficking in schizophrenia. Curr. Opin. Neurobiol. 74, 102539 (2022).
85. Pnueli, L., Rudnizky, S., Yosefzon, Y. & Melamed, P. RNA transcribed from a distal enhancer is required for activating the chromatin at the promoter of the gonadotropin a-subunit gene. Proc. Natl Acad. Sci. USA 112, 4369-4374 (2015).
86. Quinlan, A. R. & Hall, I. M. BEDTools: a flexible suite of utilities for comparing genomic features. Bioinformatics 26, 841-842 (2010).
87. Ren, C. et al. Functional annotation of structural ncRNAs within enhancer RNAs in the human genome: implications for human disease. Sci. Rep. 7, 15518 (2017).
88. Roden, D. M. et al. Development of a large-scale de-identified DNA biobank to enable personalized medicine. Clin. Pharmacol. Ther. 84, 362-369 (2008).
89. Rose, E. J. et al. Effects of a novel schizophrenia risk variant rs7914558 at CNNM2 on brain structure and attributional style. Br. J. Psychiatry 204, 115-121 (2014).
90. Sakaue, S. et al. A cross-population atlas of genetic associations for 220 human phenotypes. Nat. Genet. 53, 1415-1424 (2021).
91. Sartorelli, V. & Lauberth, S. M. Enhancer RNAs are an important regulatory layer of the epigenome. Nat. Struct. Mol. Biol. 27, 521-528 (2020).
92. Schmitt, A. D. et al. A Compendium of Chromatin Contact Maps Reveals Spatially Active Regions in the Human Genome. Cell Rep. 17, 2042-2059 (2016).
93. Sepp, M. et al. The intellectual disability and schizophrenia associated transcription factor tcf4 is regulated by neuronal activity and protein kinase A. J. Neurosci. 37, 10516-10527 (2017).
94. Shabalin, A. A. Matrix eQTL: ultra-fast eQTL analysis via large matrix operations. Bioinformatics 28, 1353-1358 (2012).
95. Siersbæk, R. et al. Transcription factor cooperativity in early adi-pogenic hotspots and super-enhancers. Cell Rep. 7, 1443-1455 (2014).

96 Sigova, A. A. et al. Transcription factor trapping by RNA in gene regulatory elements. Science 350, 978-981 (2015).
97. Stegle, O., Parts, L., Piipari, M., Winn, J. & Durbin, R. Using probabilistic estimation of expression residuals (PEER) to obtain increased power and interpretability of gene expression analyses. Nat. Protoc. 7, 500-507 (2012).
98. Sullivan, P. M. et al. The ALS/FTLD associated protein C9orf72 associates with SMCR8 and WDR41 to regulate the autophagy-lysosome pathway. Acta Neuropathol. Commun. 4, 51 (2016).

99 Trubetskoy, V. et al. Mapping genomic loci implicates genes and synaptic biology in schizophrenia. Nature 604, 502-508 (2022).

100. Tsai, P.-F. et al. A muscle-specific enhancer RNA mediates cohesion recruitment and regulates transcription in trans. Mol. Cell 71, 129-141.e8 (2018).

101. Umans, B. D., Battle, A. & Gilad, Y. Where are the disease-associated eQTLs? Trends Genet 37, 109-124 (2021).

102. Villar, D. et al. Enhancer evolution across 20 mammalian species. Cell 160, 554-566 (2015).

103. Wainberg, M. et al. Opportunities and challenges for transcriptome-wide association studies. Nat. Genet. 51, 592-599 (2019).

104. Whyte, W. A. et al. Master transcription factors and mediator establish super-enhancers at key cell identity genes. Cell 153, 307-319 (2013).

105. Xia, H. et al. Building a schizophrenia genetic network: transcription factor 4 regulates genes involved in neuronal development and schizophrenia risk. Hum. Mol. Genet. 27, 3246-3256 (2018).

106. Yamada, K. & Yoshikawa, T. From the EGR gene family to common pathways in schizophrenia: single genes versus convergent pathways. Future Neurol. 2, 347-351 (2007).

107. Yang, J. et al. Common SNPs explain a large proportion of the heritability for human height. Nat. Genet. 42, 565-569 (2010).

108. Yang, R. et al. Epiphany: predicting Hi-C contact maps from 1D epigenomic signals. Genome Biol. 24, 134 (2023).

109. Yang, Z. et al. The genome-wide risk alleles for psychiatric disorders at 3p21.1 show convergent effects on mRNA expression, cognitive function, and mushroom dendritic spine. Mol. Psychiatry 25, 48-66 (2020).

110. You, C. et al. Polygenic scores and parental predictors: an adult height study based on the united kingdom biobank and the framingham heart study. Front. Genet. 12, 669441 (2021).

111. Young, D. L. & Fields, S. The role of functional data in interpreting the effects of genetic variation. Mol. Biol. Cell 26, 3904-3908 (2015).

112. Yuan, A. et al. Effect of SOX10 gene polymorphism on early onset schizophrenia in Chinese Han population. Neurosci. Lett. 521, 93-97 (2012).

113. Zhang, S., Chasman, D., Knaack, S. & Roy, S. In silico prediction of high-resolution Hi-C interaction matrices. Nat. Commun. 10, 5449 (2019).

114. Zhang, Z. et al. HeRA: an atlas of enhancer RNAs across human tissues. Nucleic Acids Res 49, D932-D938 (2021).

115. Zheng, X., Wang, J. & Wang, C. HiCArch: A Deep Learning-based Hi-C Data Predictor. bioRxiv 2021.11.26.470146 doi.org/10.1101/2021.11.26.470146 (2021) 116. Zhou, D. et al. A unified framework for joint-tissue transcriptome-wide association and Mendelian randomization analysis. Nat. Genet. 52, 1239-1246 (2020).

117. Zhou, X. Over-representation of potential SP4 target genes within schizophrenia-risk genes. Mol. Psychiatry 27, 849-854 (2022).

118. Zou, H. & Hastie, T. Regularization and Variable Selection via the Elastic Net. J. R. Stat. Soc. Ser. B Stat. Methodol. 67, 301-320 (2005).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of assessing a subject's risk of developing a polygenic disease, the method comprising:
(a) obtaining genomic data for the subject, the genomic data comprising sequence information sufficient to determine whether the subject possesses one or more genetic variants that regulate expression of one or more enhancer RNAs (eRNAs) associated with the polygenic disease;
(b) analyzing the genomic data to determine whether the subject possesses one or more of the genetic variants;
(c) classifying the subject as having:
(i) an increased risk of the polygenic disease when one or more of the genetic variants associated with altered expression of at least one disease-associated eRNA are present, or
(ii) a baseline risk of the polygenic disease when none of the genetic variants are present.

2. The method of claim 1, wherein the classifying in step (c) further comprises: (i) computing, using a trained eRNA-based genetic risk model stored in a non-transitory computer-readable medium, a disease risk score for the polygenic disease based at least in part on the presence or absence of the genetic variants; and (ii) classifying the subject as having an increased or baseline risk of the polygenic disease based on the disease risk score.

3. The method of claim 1, further comprising imputing, using a trained tissue-specific predictive model stored in a non-transitory computer-readable medium, genetically regulated expression (GReX) values for a plurality of eRNAs from the subject's genomic data, wherein the predicted expression level is used to refine the risk classification.

4. The method of claim 3, wherein imputing the genetically regulated eRNA expression comprises applying predictive models trained on whole-genome sequencing and eRNA expression datasets across a plurality of human tissues.

5. The method of claim 3, wherein classifying the subject comprises determining whether imputed GReX values for the eRNAs meet or exceed predetermined thresholds associated with altered risk for the polygenic disease.

6. The method of claim 1, further comprising computing a disease risk score using data derived from the subject's genomic data, wherein computing the disease risk score comprises weighting the presence or absence of the genetic variants using disease-association parameters obtained from transcriptome-wide association studies (TWAS) and/or Mendelian randomization analyses.

7. The method of claim 1, wherein the polygenic disease is selected from the group consisting of: neuropsychiatric disorders, cardiometabolic disorders, hematologic traits or disorders, and sensory function disorders.

8. The method of claim 1, wherein the polygenic disease is selected from the group consisting of schizophrenia, hypertension, manifestations of mania or irritability, hearing impairment, platelet distribution width abnormality, neutrophil percentage abnormality, hemoglobin concentration abnormality, lymphocyte count abnormality, leukocyte count abnormality, cholesterol-related disorders, and obesity-related disorders.

9. The method of claim 1, wherein the polygenic disease comprises schizophrenia, the genetic variants that regulate expression of the one or more eRNAs comprise genetic variants that regulate expression of ENSR00000013481 or ENSR00000032851, and the method further comprises classifying the subject as having (i) an increased risk of schizophrenia when at least one of the genetic variants that regulate expression of ENSR00000013481 or ENSR00000032851 is present, or (ii) baseline risk of schizophrenia when none of said genetic variants is present.

10. A non-transitory computer-readable medium storing instructions that, when executed by one or more processors, cause the one or more processors to perform the method of claim 1.

11. A computer-implemented system for assessing a subject's risk of developing a polygenic disease, the system comprising:
(a) at least one processor; and
(b) a non-transitory computer-readable storage medium storing instructions which, when executed by the at least one processor, cause the system to:
(i) receive genomic data for the subject, the genomic data comprising sequence information sufficient to determine whether the subject possesses one or more genetic variants that regulate expression of one or more enhancer RNAs (eRNAs);
(ii) analyze the genomic data to determine whether the subject possesses one or more of the genetic variants that regulate expression of the one or more eRNAs; and
(iii) classify the subject as having an increased or baseline risk of the polygenic disease based at least in part on whether one or more of the genetic variants that regulate expression of a polygenic-disease-associated eRNA are present in the genomic data.

12. The system of claim 11, wherein the instructions further cause the system to:
(iv) compute, using a trained eRNA-based genetic risk model stored in the non-transitory computer-readable storage medium, a disease risk score for the polygenic disease based at least in part on the presence or absence of the genetic variants; and
(v) classify the subject as having an increased or baseline risk of the polygenic disease based on the disease risk score.

13. The system of claim 11, wherein the instructions stored in the non-transitory computer-readable storage medium further cause the system to impute, using a trained tissue-specific predictive model, genetically regulated expression (GReX) values for a plurality of eRNAs from the subject's genomic data.

14. The system of claim 13, wherein imputing the genetically regulated eRNA expression comprises applying predictive models trained on whole-genome sequencing and eRNA expression datasets across a plurality of human tissues.

15. The system of claim 11, wherein the instructions further cause the system to compute a disease risk score for the polygenic disease using data derived from the subject's genomic data, and wherein computing the disease risk score comprises weighting the presence or absence of the genetic variants using disease-association parameters obtained from transcriptome-wide association studies (TWAS) and/or Mendelian randomization analyses.

16. The system of claim 11, wherein the polygenic disease is selected from the group consisting of: neuropsychiatric disorders, cardiometabolic disorders, hematologic traits or disorders, and sensory function disorders.

17. The system of claim 11, wherein the polygenic disease is selected from the group consisting of schizophrenia, hypertension, manifestations of mania or irritability, hearing impairment, platelet distribution width abnormality, neutrophil percentage abnormality, hemoglobin concentration abnormality, lymphocyte count abnormality, leukocyte count abnormality, cholesterol-related disorders, and obesity-related disorders.

18. The system of claim 11, wherein the polygenic disease comprises schizophrenia, and wherein the instructions further cause the system to: (i) determine, from the genomic data, whether the subject possesses one or more genetic variants that regulate expression of eRNAs ENSR00000013481 or ENSR00000032851; and (ii) classify the subject as having increased schizophrenia risk when at least one of said genetic variants is present and baseline schizophrenia risk when none of said genetic variants is present.

19. The system of claim 18, wherein the instructions stored in the non-transitory computer-readable storage medium further cause the system to:
(i) impute, using a trained brain-specific predictive model, genetically regulated expression (GReX) values for eRNAs ENSR00000013481 and/or ENSR00000032851 from the subject's genomic data; and
(ii) refine the subject's schizophrenia risk classification based at least in part on the imputed GReX values for ENSR00000013481 and/or ENSR00000032851.

20. The system of claim 11, wherein the instructions further cause the system to compute a disease risk score for schizophrenia using data derived from the subject's genomic data, and wherein computing the disease risk score comprises weighting the presence or absence of the genetic variants that regulate expression of ENSR00000013481 or ENSR00000032851 using disease association parameters obtained from transcriptome-wide association studies (TWAS) and/or Mendelian randomization analyses.
